(19) United States
(12) Patent Application Publication
(10) Pub. No.: US 2006/0047196 A1
(43) Pub. Date: Mar. 2, 2006

(54) Method and apparatus for magnetic resonance analysis

(75) Inventors: Ehud Katzenelson, Ramat Ishai (IL); Uzi Dan, Ganei Yehuda (IL); Alex Harel, Savyon (IL)

Correspondence Address: Martin Moynihan;Anthony Castorina, Suite 207, 2001 Jefferson Davis Highway, Arlington, VA, 22202, US

(73) Assignee: BBMS Ltd., Savyon (IL)

(21) Appl. No.: 10/531,243

(22) PCT Filed: Oct. 19, 2003
(86) PCT No.: PCT/IL03/00845

§ 371 Date: Apr. 14, 2005

Related U.S. Application Data
(60) Provisional application No. 60/419,588, filed on Oct. 21, 2002.
(60) Provisional application No. 60/433,577, filed on Dec. 16, 2002.

(51) Int. Cl. A61B 5/05

(52) U.S. Cl. 600/410

(57) ABSTRACT

A method of designing a magnetic structure for providing a monotonic static magnetic field for magnetic resonance analysis. The method comprises: selecting a first geometry defining a volume-of-interest and selecting a magnetic field query, which is defined on a plurality of coordinates within the first geometry, the magnetic field query being monotonic. The method further comprises selecting a second geometry defining the magnetic structure and calculating a remanence distribution within the second geometry, by using the first geometry, the second geometry and the magnetic field query.

### FIELD OF THE INVENTION

The present invention relates to magnetic resonance analysis and, more particularly, to a magnet for generating a substantially non-homogenous magnetic field for the purpose of magnetic resonance analysis. The present invention further relates to a method of designing the magnet.

### BACKGROUND OF THE INVENTION

Magnetic Resonance Imaging (MRI) is a method to obtain an image representing the chemical and physical microscopic properties of materials, by utilizing a quantum mechanical phenomenon, known as Nuclear Magnetic Resonance (NMR), in which a system of spins, placed in a magnetic field resonantly absorb energy, when applied with a certain frequency.

A nucleus can experience NMR only if its nuclear spin I does not vanish, i.e., the nucleus has at least one unpaired nucleon. Examples of non-zero spin nuclei frequently used in MRI include 1H (I=1/2), ²H (I=1), ²³Na (I=3/2), etc. When placed in a magnetic field, a nucleus having a spin I is allowed to be in a discrete set of energy levels, the number of which is determined by I, and the separation of which is determined by the gyromagnetic ratio of the nucleus and by the magnetic field. Under the influence of a small perturbation, manifested as a radiofrequency (RF) magnetic field, which rotates about the direction of a primary static magnetic field, the nucleus has a time dependent probability to experience a transition from one energy level to another. With a specific frequency of the rotating magnetic field, the transition probability may reach the value of unity. Hence at certain times, a transition is forced on the nucleus, even though the rotating magnetic field may be of small magnitude relative to the primary magnetic field. For an ensemble of spin I nuclei the transitions are realized through a change in the overall magnetization.

Once a change in the magnetization occurs, a system of spins tends to restore its magnetization longitudinal equilibrium value, by the thermodynamic principle of minimal energy. The time constant which control the elapsed time for the system to return to the equilibrium value is called “spin-lattice relaxation time” or “longitudinal relaxation time” and is denoted T₁. An additional time constant, T₂ (≦T₁), called “spin-spin relaxation time” or “transverse relaxation time”, controls the elapsed time in which the transverse magnetization diminishes, by the principle of maximal entropy. However, inter-molecule interactions and local variations in the value of the static magnetic field alter the value of T₂, to an actual value denoted T₂*.

In MRI systems the static magnetic field is generated by a main magnet. A primary requirement from prior art main magnets is that the generated field be uniform. For example, in clinical imaging application, typical homogeneities are of the order of few parts per million over a spherical volume of 50 cm. Depending on the application, permanent, resistive or superconducting magnets are used in MRI systems. In most MRI systems to date a large magnet which effectively surrounds the patient is employed. Such magnets are typically large superconductor magnets which are expensive but are unavoidable when whole body imaging is required. However, when only local imaging of small sections of body tissue is required, it becomes possible to use more compact arrangements employing smaller magnets. Permanent magnets offer the advantage of simplicity and affordability, and, in addition, these magnets may also be relatively compact.

MRI modalities, as well as many localized spectroscopic techniques require a static magnetic field having a predetermined gradient, so that a unique magnetic field is generated at each region of the analyzed object. By detecting the NMR signal knowing the magnetic field gradient, the position of each region of the object can be imaged. In typical MRI systems gradients in predetermined directions are obtained by providing additional coils which generate the desired gradients. Gradient coils naturally add complexity to the MRI system. For example, for producing gradients in the three spatial directions without physically rotating the gradient coils, three gradient coils are used.

The number of gradient coils which are used dictates the complexity of procedures like balancing and tuning. In addition, gradient coils are characterized by a natural self-inductance, which results in their inability to be switched on and off instantaneously. Thus, during the periods of building up and decay of the currents within the gradient coils, the temporal change of the magnetic flux, originally generated by the currents, creates eddy currents in the surrounding structure. The eddy currents generate secondary a magnetic fields which may interfere with the primary gradient fields hence affect the imaging accuracy.

Furthermore, in MRI systems using permanent magnets, if the gradient coils are positioned in close proximity to the permanent magnets, the heat developed in the resistive gradient coils by the currents flowing within the coils may heat the permanent magnet, resulting in a local temperature increase. Such temperature changes are undesirable since the field generated by permanent magnets is highly susceptible to large variations induced by local temperature changes.

The present invention provides solutions to the problems associated with prior art MRI techniques.

### SUMMARY OF THE INVENTION

According to one aspect of the present invention there is provided a method of designing a magnetic structure for providing a monotonic static magnetic field for magnetic resonance analysis, the method comprising: selecting a first geometry defining a volume-of-interest; selecting a magnetic field query, defined on a plurality of coordinates within the first geometry, the magnetic field query being monotonic; selecting a second geometry defining the magnetic structure; and calculating a remanence distribution within the second geometry by using the first geometry, the second geometry and the magnetic field query, thereby designing the magnetic structure.

According to further features in preferred embodiments of the invention described below, the method further comprising optimizing the monotonic static magnetic field, by repeating the selecting the first and second geometries and the magnetic field query and repeating the calculating of the remanence distribution.

According to still further features in the described preferred embodiments the remanence distribution is calculated by constructing a functional of the remanence distribution and minimizing the functional using a set of constraints.

According to still further features in the described preferred embodiments the remanence distribution is calculated by constructing a functional of the remanence distribution and maximizing the functional using a set of constraints.

According to still further features in the described preferred embodiments each constraint of the set of constraints is selected from the group consisting of an equality constraint and an inequality constraint.

According to still further features in the described preferred embodiments the set of constraints are selected so as to optimize the monotonic static magnetic field.

According to another aspect of the present invention there is provided a magnetic structure for magnetic resonance analysis, comprising a structure defined according to a remanence distribution, the remanence distribution being determined according to a first geometry for defining a volume-of-interest, a second geometry for defining the magnetic structure, and a magnetic field query being defined on a plurality of coordinates within the first geometry, the magnetic field query being monotonic.

According to further features in preferred embodiments of the invention described below, the first geometry is selected so that a maximal value of at least one component of the magnetic field query is above a predetermined threshold.

According to still further features in the described preferred embodiments the second geometry is selected so that a maximal value of at least one component of the magnetic field query is above a predetermined threshold.

According to still further features in the described preferred embodiments the predetermined threshold is selected so as to optimize a signal-to-noise ratio and a signal to contrast.

According to still another aspect of the present invention there is provided a magnetic structure for magnetic resonance analysis, the magnetic structure comprising a plurality of domains, arranged within a volume having predetermined geometry, each of the plurality of domains being characterized by a predetermined and different magnetization vector; wherein the predetermined geometry and the plurality of domains are selected so as to generate a monotonic static magnetic field having a gradient.

According to further features in preferred embodiments of the invention described below, the magnetic structure further comprising at least one additional magnetic structure designed connectable to the magnetic structure, wherein the at least one additional magnetic structure capable of generating a monotonic static magnetic field.

According to still further features in the described preferred embodiments the magnetic structure further comprising at least one non-magnetic domain located so as to optimize a profile of the monotonic static magnetic field.

According to still further features in the described preferred embodiments the magnetic structure is designed connectable to a radiofrequency antenna and the at least one non-magnetic domain is constructed and designed for minimizing a load on the radiofrequency antenna and for minimizing magnetic acoustic ringing.

According to vet another aspect of the present invention there is provided an apparatus for magnetic resonance analysis, the apparatus comprising: a processing unit; a radiofrequency coil designed and configured for generating a broad-band radiofrequency magnetic field; and a magnetic structure for generating a monotonic static magnetic field having a gradient, the magnetic structure comprising a plurality of domains, the plurality of domains being arranged within a volume having predetermined geometry, each of the plurality of domains being characterized by a predetermined and different magnetization vector, wherein the predetermined geometry and the plurality of domains are selected so as to generate the monotonic static magnetic field.

According to further features in preferred embodiments of the invention described below, the apparatus further comprising a first gradient coil for generating a magnetic field having a gradient substantially in a first transverse direction.

According to still further features in the described preferred embodiments the apparatus further comprising a second gradient coil for generating a magnetic field having a gradient substantially in a second transverse direction.

According to still further features in the described preferred embodiments the apparatus further comprising at least one additional magnetic structure designed connectable to the magnetic structure, wherein the at least one additional magnetic structure capable of generating a monotonic static magnetic field.

According to still further features in the described preferred embodiments the apparatus further comprising at least one additional radiofrequency coil for generating a broad-band radio frequency magnetic field and at least one additional magnetic structure for generating a monotonic static magnetic field having a gradient, wherein each of the at least one additional radiofrequency coil is in proximity to the at least one additional magnetic structure.

According to still further features in the described preferred embodiments the apparatus further comprising a power supply and a wireless transmitter for transmitting information from the radiofrequency coil, wherein a size of the radiofrequency coil and a size of the magnetic structure are selected so as to capsule the radiofrequency coil and the magnetic structure into a compact probe to be swallowed by a subject.

According to an additional aspect of the present invention there is provided a system for analyzing an object, the system comprising: a processing unit; a first imaging device; and a magnetic resonance probe, the magnetic resonance probe comprising a radiofrequency coil, designed and configured for generating a broad-band radiofrequency magnetic field, and a magnetic structure for generating a monotonic static magnetic field having a gradient, the magnetic structure comprising a to plurality of domains, the plurality of domains being arranged within a volume having predetermined geometry, each of the plurality of domains being characterized by a predetermined and different magnetization vector, wherein the predetermined geometry and the plurality of domains are selected so as to generate the monotonic static magnetic field.

According to further features in preferred embodiments of the invention described below, the object is an internal object and the system is an invasive system.

According to still further features in the described preferred embodiments the object is an external object and the system a non-invasive system.

According to still further features in the described preferred embodiments the first imaging device is an optical imaging device.

According to still further features in the described preferred embodiments the optical imaging device is a camera.

According to still further features in the described preferred embodiments the first imaging device is an ultra-sonic imaging device.

According to still further features in the described preferred embodiments the first imaging device is a nuclear medicine device.

According to still further features in the described preferred embodiments the system further comprising at least one additional imaging device, the at least one additional imaging device is selected from the group consisting of an optical imaging device, a US imaging device and a nuclear medicine device.

According to still further features in the described preferred embodiments the first imaging device has a sufficiently wide field-of-view so as to surround at least a portion of the magnetic resonance probe.

According to still further features in the described preferred embodiments the system further comprising a communication cable connected to the first imaging device.

According to still further features in the described preferred embodiments the system further comprising at least one supporting device for supporting the magnetic resonance probe and the first imaging device.

According to still further features in the described preferred embodiments the system further comprising a position tracking system for determining a position of the magnetic resonance probe.

According to still further features in the described preferred embodiments a size of the first imaging device and a size of the magnetic resonance probe are selected so as to allow the first imaging device and the magnetic resonance probe to be inserted into a body of a subject by endoscopy.

According to still further features in the described preferred embodiments a size of the first imaging device, a size of the magnetic resonance probe and sizes of the at least one imaging device are selected so as to allow the first imaging device, the magnetic resonance probe and the at least one imaging device to be inserted into a body of a subject by endoscopy.

According to yet an additional aspect of the present invention there is provided a system for analyzing an object, the system comprising: a processing unit; a magnetic resonance probe; and a position tracking system for determining a position of the magnetic resonance probe; wherein the magnetic resonance probe comprising a radiofrequency coil, designed and configured for generating a broad-band radiofrequency magnetic field, and a magnetic structure for generating a monotonic static magnetic field having a gradient, the magnetic structure comprising a plurality of domains, the plurality of domains being arranged within a volume having predetermined geometry, each of the plurality of domains being characterized by a predetermined and different magnetization vector, wherein the predetermined geometry and the plurality of domains are selected so as to generate the monotonic static magnetic field.

According to further features in preferred embodiments of the invention described below, the position tracking system is selected from the group consisting of an articulated arm position tracking system, an accelerometers based position tracking system, a potentiometers based position tracking system, a sound wave based position tracking system, a radio frequency based position tracking system, an AC based position tracking system, a magnetic field based position tracking system and an optical based position tracking system.

According to still further features in the described preferred embodiments the system further comprising a first gradient coil for generating a magnetic field having a gradient substantially in a first transverse direction.

According to still further features in the described preferred embodiments the system further comprising a second gradient coil for generating a magnetic field having a gradient substantially in a second transverse direction.

According to still further features in the described preferred embodiments the first gradient coil is positioned on a surface of the magnetic structure.

According to still further features in the described preferred embodiments the first and the second gradient coils are positioned on a surface of the magnetic structure.

According to still further features in the described preferred embodiments the first and the second gradient coils are arranged in one layer.

According to still further features in the described preferred embodiments the first and the second gradient coils are arranged separate layers.

According to still further features in the described preferred embodiments the radiofrequency coil is positioned on a surface of the magnetic structure and further wherein the first gradient coil and the radiofrequency coil are arranged in one layer.

According to still further features in the described preferred embodiments the magnetic structure is detachable.

According to still further features in the described preferred embodiments the magnetic structure is replaceable.

According to still further features in the described preferred embodiments the magnetic structure comprises at least two parts, each independently operable to rotate about a longitudinal axis.

According to still further features in the described preferred embodiments the magnetic structure comprises at least two parts, each independently operable to move along a longitudinal axis.

According to still further features in the described preferred embodiments the plurality of domains comprises at least three domains.

According to still further features in the described preferred embodiments the plurality of domains comprises at least four domains.

According to still further features in the described preferred embodiments the predetermined geometry is selected from the group consisting of a cylinder, a disk, a prism, a sphere, a hemisphere, a portion of a sphere, an ellipsoid, a portion of ellipsoid, a hyperboloid, a portion of a hyperboloid, a paraboloid, a portion of a paraboloid a cylindrical shell, a portion of a cylindrical shell, a polyhedron shell and a portion of a polyhedron shell.

According to still further features in the described preferred embodiments the predetermined geometry is elongated with respect to a longitudinal axis.

According to still further features in the described preferred embodiments the plurality of domains are arranged along the longitudinal axis, and further wherein a magnetization vector of each domain has a component directed perpendicular to the longitudinal axis so that the monotonic static magnetic field is also in a direction perpendicular to the longitudinal axis.

According to still further features in the described preferred embodiments the plurality of domains are arranged along the longitudinal axis, and further wherein a magnetization vector of each domain has a component directed parallel to the longitudinal axis so that the monotonic static magnetic field is also in a direction parallel to the longitudinal axis.

According to still further features in the described preferred embodiments the magnetic structure and the radiofrequency coil is designed and constructed so that the monotonic static magnetic field and the radiofrequency magnetic field are capable of generating predetermined and different magnetic resonance responses in predetermined and different types of cells.

According to still further features in the described preferred embodiments the predetermined types of cells are selected from the group consisting of a part of a tumor, a part of a malignant tumor, a part of a blood vessel tissue, a part of a pathological tissue and a part of a restenotic tissue.

According to still further features in the described preferred embodiments the magnetic structure and the radiofrequency coil are designed and constructed so that the monotonic static magnetic field and the radiofrequency magnetic field are capable of generating a predetermined and different magnetic resonance responses in a first substance and in at least one additional substance present in or surrounded by the first substance, thereby distinguishing between the first substance and the at least one additional substance.

According to still further features in the described preferred embodiments the magnetic structure and the radiofrequency coil are designed and constructed so that the monotonic static magnetic field and the radiofrequency magnetic field are capable of generating a magnetic resonance response in at least one substance having dynamically resonance characteristics, the dynamically resonance characteristics being time-dependent.

According to still further features in the described preferred embodiments the system and the apparatus are design and constructed to monitor the dynamically resonance characteristics.

According to still further features in the described preferred embodiments the system further comprising at least one additional magnetic structure designed connectable to the magnetic structure, wherein the at least one additional magnetic structure capable of generating a monotonic static magnetic field.

According to still further features in the described preferred embodiments the at least one additional magnetic structure operable to rotate about a transverse axis by one of a plurality of predetermined angles.

According to still further features in the described preferred embodiments the monotonic static magnetic field varies along the longitudinal axis.

According to still further features in the described preferred embodiments the magnetization vectors vary along a radial direction, the radial direction being perpendicular to the longitudinal axis.

According to still further features in the described preferred embodiments the magnetization vectors vary along an azimuthal direction, the azimuthal direction being perpendicular to the longitudinal axis.

According to still further features in the described, preferred embodiments the magnetic resonance probe further comprising at least one non-magnetic domain located so as to optimize a profile of the monotonic static magnetic field.

According to still further features in the described preferred embodiments the radiofrequency coil comprises a radiofrequency antenna and the at least one non-magnetic domain is constructed and designed for minimizing a load on the radiofrequency antenna and for minimizing magnetic acoustic ringing.

According to still further features in the described preferred embodiments a size of the at least one non-magnetic domain is selected so as to be surrounded by at least one radiofrequency coil.

According to still further features in the described preferred embodiments a size of the at least one non-magnetic domain is selected so as to minimize an amount of magnetic material present in the magnetic structure.

According to still further features in the described preferred embodiments the at least one radiofrequency coil comprises a soft magnetic material.

According to still further features in the described preferred embodiments the predetermined geometry is characterized by at least one substantially planar surface, the at least one substantially planar surface defined by an axis being perpendicularly to the at least one substantially planar surface.

According to still further features in the described preferred embodiments the plurality of domains are concentrically arranged about a center of the magnetic structure, and further wherein a magnetization vector of each domain has a component directed along the axis, so that the monotonic static magnetic field is also directed along the axis.

According to still further features in the described preferred embodiments the predetermined geometry is characterized by a planar surface and a non-planar surface, the non-planar surface having a first open end, adjacent to the planar surface, and a second open end, far from the planar surface, where an area of the first open end is smaller than an area of the second open end.

According to still further features in the described preferred embodiments the plurality of domains are arranged within the non-planar surface, and further wherein a magnetization vector of each domain has a component directed substantially parallel to the planar surface, so that the monotonic static magnetic field is also directed substantially parallel to the planar surface.

According to still further features in the described preferred embodiments the predetermined geometry is a shell having a cavity land a symmetry axis, the shell is selected from the group consisting of a hemisphere, a portion of a sphere, an ellipsoid, a portion of ellipsoid, a hyperboloid, a portion of a hyperboloid, a paraboloid, a portion of a paraboloid, a cylindrical shell, a portion of a cylindrical shell, a polyhedron shell and a portion of a polyhedron shell.

According to still further features in the described preferred embodiments a magnetization vector of each domain is directed along the symmetry axis so that the monotonic static magnetic field is also directed along the symmetry axis.

According to still further features in the described preferred embodiments a magnetization vector of each domain is directed perpendicularly to the symmetry axis so that the monotonic static magnetic field is also perpendicularly along the symmetry axis.

According to still further features in the described preferred embodiments the system further comprising at least one additional radiofrequency coil for generating a broad-band radiofrequency magnetic field and at least one additional magnetic structure for generating a monotonic static magnetic field having a gradient, wherein each of the at least one additional radiofrequency coil is in proximity to the at least one additional magnetic structure.

According to still further features in the described preferred embodiments the object is a mammal.

According to still further features in the described preferred embodiments the object is an organ of a mammal.

According to still further features in the described preferred embodiments the object is a tissue.

According to still further features in the described preferred embodiments the object is a swollen elastomer.

According to still further features in the described preferred embodiments the object is a food material.

According to still further features in the described preferred embodiments the object is liquid.

According to still further features in the described preferred embodiments the liquid is oil.

According to still further features in the described preferred embodiments the object is at least one type of molecules present in a solvent.

According to still further features in the described preferred embodiments the at least one type of molecules present in the solvent is selected from the group consisting of molecule dissolved in the solvent, a molecule dispersed in the solvent and a molecule emulsed in the solvent.

The present invention successfully addresses the shortcomings of the presently known configurations by providing a method and apparatus for magnetic resonance analysis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods and examples are illustrative only and not intended to be limiting.

Implementation of the method and system of the present invention involves performing or completing selected tasks or stages manually, automatically, or a combination thereof. Moreover, according to actual instrumentation and equipment of preferred embodiments of the method and system of the present invention, several selected stages could be implemented by hardware or by software on any operating system of any firmware or a combination thereof. For example, as hardware, selected stages of the invention could be implemented as a chip or a circuit. As software, selected stages of the invention could be implemented as a plurality of software instructions being executed by a computer using any suitable operating system. In any case, selected stages of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows a flowchart of a method of designing a magnetic structure for providing a monotonic static magnetic field, according to a preferred embodiment of the present invention;

FIG. 2 is a schematic illustration of a magnetic structure for magnetic resonance analysis, according to a preferred embodiment of the present invention;

FIG. 3 is a schematic illustration of an elongated magnetic structure, according to a preferred embodiment of the present invention;

FIG. 4 is a schematic illustration of a combination of two magnetic structures, according to a preferred embodiment of the present invention;

FIGS. 5a-c are simplified illustrations of a magnetic structure having a planar surface, according to a preferred embodiment of the present invention;

FIG. 6 is a schematic illustration of a magnetic structure shaped as a shell, according to a preferred embodiment of the preset invention;

FIG. 7 is a schematic illustration of an apparatus for magnetic resonance analysis, according to a preferred embodiment of the present invention;

FIG. 8 is a schematic illustration of an apparatus for magnetic resonance analysis, which comprises at least one additional magnetic probe, according to a preferred embodiment of the present invention;

FIGS. 9a-b show schematic illustrations of a system for analyzing an object, which comprises a magnetic resonance probe and an imaging device, according to a preferred embodiment of the present invention;

FIG. 10 is a schematic illustration of a system for analyzing an object, which comprises a magnetic resonance probe and a position tracking system, according to a preferred embodiment of the present invention;

FIG. 11a shows the magnetizations of each domain of a cylindrical magnet, and the generated radial magnetic field as a function of the distance from the center the cylinder;

FIG. 11b shows the magnetizations of each domain of a cylindrical magnet, and the generated axial magnetic field as a function of the distance from the center the cylinder;

FIG. 12 shows the magnetizations of each domain of a disk magnet and the magnetic field as a function of the distance from the surface of the disk; and

FIG. 13 shows the magnetizations of each domain of a hemisphere magnet and the magnetic field as a function of the distance from the surface of the hemisphere.

### DESCRIPTION OF THE PREFERRED EMBODIMENTS

The present invention is of a magnetic structure which can preferably be used for the purpose of magnetic resonance analysis, such as MRI, NMR and the like. Specifically and more preferably, the present invention is of a magnetic structure which can be used to generate a substantially non-homogenous magnetic field in an MRI apparatus. The present invention is further of a method for of designing the magnetic structure, an apparatus for magnetic resonance analysis incorporating the magnetic structure and a system for analyzing an object incorporating the apparatus is for magnetic resonance analysis.

The principles and operation of an exemplary magnetic structure according to the present invention and a method of designing the same according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

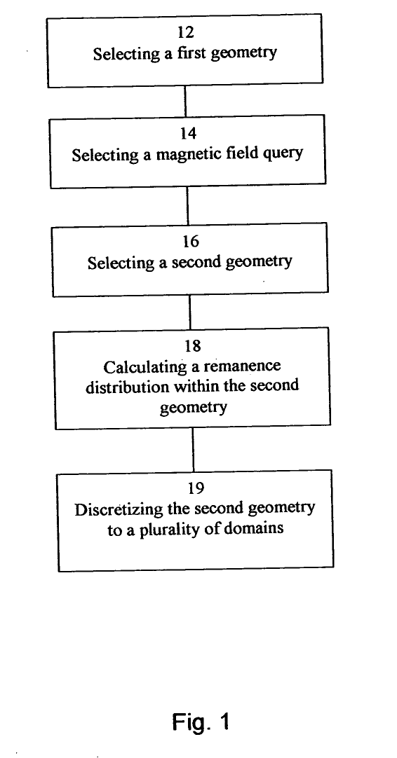

Referring now to the drawings, FIG. 1 shows a flowchart of a method of designing a magnetic structure for providing a monotonic static magnetic field for magnetic resonance analysis. The method comprises the following method stages in which in a first stage, designated by Block 12, a first geometry which defines a volume-of-interest is selected. The selected volume-of-interest is preferably the region in which the magnetic resonance analysis is to be executed, e.g., by an appropriate system or apparatus. Thus, for example, the volume of interest may be a region in close proximity to the magnetic structure, or a region surrounded by at least a portion of the magnetic structure.

In a second stage, designated by Block 14, a monotonic magnetic field query is selected, which magnetic field query is defined on a plurality of coordinates within the first geometry. The magnetic field query is the desired static magnetic field which is to be present within the volume-of-interest selected in the first stage.

In a third stage, designated by Block 16, a second geometry which defines the magnetic structure is selected. The second geometry is preferably the desired shape of the magnetic structure, which shape is selected according to the specific application of the magnetic structure and according to the desired static magnetic field.

According to a preferred embodiment of the present invention, once the magnetic field query and the second geometry are is selected, a maximal value of at least one of the components of the magnetic field query is calculated, preferably as a volume integral of a predetermined Green's function, over the entire second geometry. A preferred expression for the Green's function is:

$$\begin{matrix}{G}_{i , k} ( x , {x}_{1} ) = {E}_{j , k} ( x - {x}_{1} ) \\ where , \\ {E}_{i , k} ( x ) = \frac{1}{4 π {μ}_{0} {\vert x \vert}^{5}} ( 3 {x}_{i} {x}_{k} - {\vert x \vert}^{2} {δ}_{i , k} ) .\end{matrix} \qquad ( EQ . 1 )$$

Thus, the second geometry is preferably selected so that the maximal value of the magnetic field query is above a predetermined threshold, which ensures a substantially optimized signal-to-noise ratio.

According to a preferred embodiment of the present invention the second stage may be executed more than once so as to obtain the desired magnetic filed characteristics, e.g., a desired signal-to-noise ratio and/or a desired contrast-to-noise ratio suitable for the specific application for which the magnetic structure is designed (e.g., imaging of specific types of cells or substrates). In other words, if, for example, a certain shape was selected for the magnetic structure, and it was found that for this particular shape the maximal value of the magnetic field is below the predetermined threshold, then the second stage is repeated and a new shape/geometry of the magnetic structure is selected. This process is iteratively repeated until the maximal value of the magnetic field is satisfactory.

In a fourth stage designated by Block 18, a remanence distribution, J(x), is calculated within the second geometry, where the calculation is based on the first geometry, the second geometry and the magnetic field query. Remanence is known as the intercept of the B-H curve with the H=0 axis, where H and B are the magnetic field vectors generated by free currents and total currents within the magnetic structure, respectively. It is convenient to define the remanence distribution in terms of the magnetization, M(x), of the magnetic structure:
J(x)=μ₀M(x), (EQ. 2)
where μ₀ is the vacuum permeability.

According to a preferred embodiment of the present invention the remanence distribution, J(x), is calculated by two substages. Hence, in a first substage, a functional, U₀, of the remanence distribution, J, is constructed, and in a second substage, the functional, U₀, is either minimized or maximized depending on the choice of U₀. For example, one possible expression for U₀, for which minimization is used, may be:

$${U}_{0} \{ J ( x ) \} = \frac{1}{2} \int ⅆ V \vert J ( x ) \cdot J ( x ) \vert . \qquad ( EQ . 3 )$$

It is to be understood, however, that other definitions for the functional U₀ are within the scope of the present invention, provided that it has an extremum in which the remanence distribution (or magnetization) corresponds to a monotonic static magnetic field. These other definitions include, for example other moments of J or combination between two or more moments. The minimization or maximization of U₀ is by variation calculus, preferably under a set of constraints, which is related to the characteristics of the query magnetic field. In other words, one seeks a solution to the problem of finding an extremum to U₀{J(x)}, given a predetermined magnetic effect of the remanence distribution, J, on the predetermined volume-of-interest. Specifically, if one assumes that the magnetization is substantially not effected by the magnetic field it generates and that the norm of J is substantially uniform throughout the second geometry, then, once J is found, the jth component of the magnetic field is given by:
Bⱼ(x)=∫dV Gⱼₖ(x) Jₖ(x), (EQ. 4)
where here and throughout the specification, the Einstein summation convention over repeated indices is employed.

According to a preferred embodiment of the present invention each constraint of the set of constrains may be either equality or inequality constraint. The constraints are preferably are selected so as to optimize the magnetic field B. For example, the constraints may be imposed on one or more components of the magnetic field B and/or on one or more of its spatial derivatives. A detailed example of a set of constraints, according to a preferred embodiment of the present invention, and the corresponding equations is given in the Examples section that follows.

One would appreciate, however, that for a particular combination of the volume-of-interest, the magnetic structure shape and the magnetic field query, there may be no mathematical solution to the extremum problem, or that the resulting B, generated by the obtained remanence distribution is not optimized (e.g., insufficient signal-to-noise ratio). In this case the procedure is optionally and preferably repeated iteratively by changing volume-of-interest, the magnetic structure shape and the magnetic field query in any combination, until an optimal solution is found.

The obtained remanence distribution is continues function over the entire volume of the magnetic structure. Industrial manufacturing processes, however, are known to favor a remanence distribution which is composed of a discrete set of local remanence distributions, where each local remanence distribution corresponds to one domain within the magnetic structure. Hence, according to a preferred embodiment of the present invention, the method comprises a fifth stage designated by Block 19, in which the second geometry is discretized to a plurality of domains. Then, the remanence distribution which corresponds to the plurality of domains is preferably recalculated for the domains to obtain the final and desired remanence distribution.

Performing the above method according to present invention successfully produces a magnetic structure which is designed according to the above method stages. Therefore, according to another aspect of the present invention, there is provided a magnetic structure for magnetic resonance analysis. The magnetic structure comprises a structure defined according to a remanence distribution, which is determined according to the first geometry, the second geometry and the monotonic magnetic field query, as further detailed hereinabove.

According to an additional aspect of the present invention there is provided a magnetic structure for magnetic resonance analysis, generally referred to herein as magnetic structure 20.

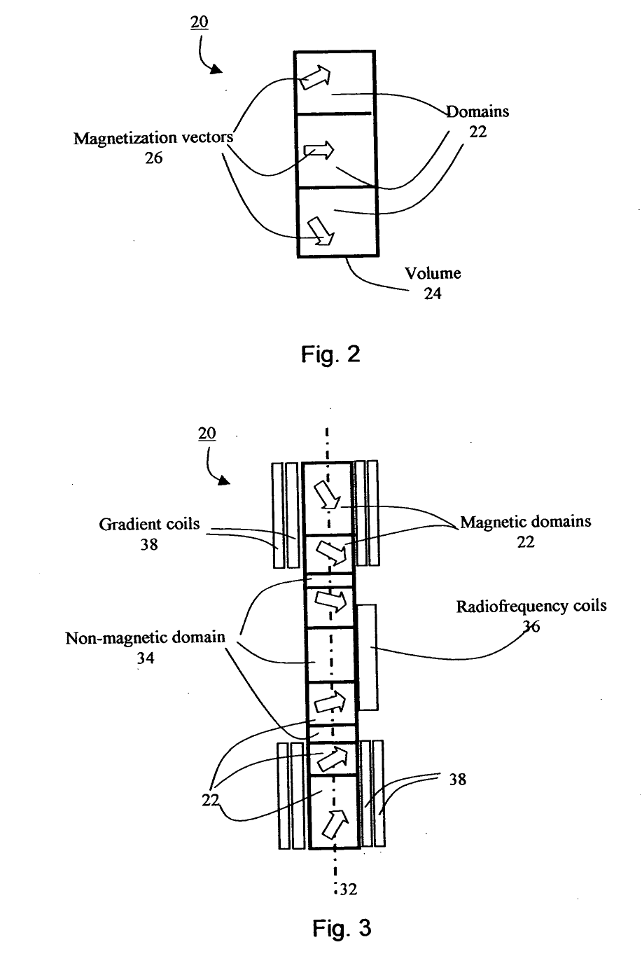

Reference is now made to FIG. 2, which is a schematic illustration of magnetic structure 20. Hence, magnetic structure 20 comprises a plurality of domains 22, arranged within a volume 24 having predetermined geometry. Each domain is characterized by a predetermined and different magnetization vector 26. The geometry and domains 22 are selected so as to generate a monotonic static magnetic field, B, having a gradient.

A typical gradient value of the generated magnetic field is from few Gauss/cm up to 10,000 Gauss/cm. However, it is expected that during the life of this patent many relevant magnetic resonance analysis applications employing non-homogenous magnetic fields will be developed and the scope of the phrase “a gradient” is intended to include all such new technologies a priori.

The geometry of volume 24 is preferably selected in accordance with the specific application to which magnetic structure 20 is employed. If, for example, magnetic structure 20 is employed in a magnetic resonance analysis device which is typically used to analyze objects which are near flat surfaces, the geometry of volume 24 as selected is preferably substantially flat. If, on the other hand, magnetic structure 20 is employed in an endoscopic device, the geometry is preferably selected so as to facilitate the endoscopy procedure (e.g., elongated and thin geometry). It is not intended, however, to limit the scope of the present invention to any specific geometry of volume 24. Thus, volume 24 may optionally have any suitable geometry including, but not limited to, a cylinder, a disk, a prism, a sphere, a hemisphere, a portion of a sphere, an ellipsoid, a portion of ellipsoid, a hyperboloid, a portion of a hyperboloid, a paraboloid or a portion of a paraboloid, a cylindrical shell, a portion of a cylindrical shell, a polyhedron or a portion of a polyhedron.

Reference is now made to FIG. 3 which is a schematic illustration of magnetic structure 20 in which the geometry of volume 24 is elongated with respect to a longitudinal axis 32, according to a preferred embodiment of the present invention. As stated, such a design may optionally be used, for example, for an endoscopy procedure, in which case the size of magnetic structure 20 is preferably selected so as to allow the magnetic structure to be to be inserted into a body of a subject.

Domains 22 may be arranged long longitudinal axis 32, in more than one way. For example, in one embodiment each one of magnetization vectors 26 has a component which is directed perpendicular to longitudinal axis 32, so that the static magnetic field, B, is generated also in a direction perpendicular to longitudinal axis 32. In another embodiment, each one of magnetization vectors 26 has a component directed parallel to longitudinal axis 32 so that the static magnetic field, B, is directed parallel to longitudinal axis 32.

Whether the static magnetic field, B, is directed parallel or perpendicular to longitudinal axis 32, both the magnitude and the direction of magnetization vectors 26 may vary along any direction, so as to provide further control on the generated magnetic field and its gradient. Specifically, for each one of domains 22, magnetization vectors 26 may vary along longitudinal axis 32 and/or along any direction which is perpendicular thereto, e.g., a radial direction and an azimuthal direction.

According to a preferred embodiment of the present invention, magnetic structure 20 preferably comprises at least one non-magnetic domain 34 which is located within magnetic structure 20 so as to optimize a profile of the magnetic field, B. In other words, in this embodiment magnetic structure 20 is preferably composed of a combination of magnetic and non-magnetic domains, such that the magnetic domains are characterized by non-zero magnetization vectors, while in the non-magnetic domains the magnetization substantially vanishes.

As the present invention is primarily directed at providing a magnetic structure to be used in a magnetic resonance analysis device, magnetic structure 20 is preferably combined with at least one radiofrequency coil 36. Radiofrequency coil 36 may be located on each of the surfaces of magnetic structure 20. Specifically, radiofrequency coil 36 may surround magnetic structure 20 and/or located on a particular side (top side, bottom side, etc.) of magnetic structure 20. In another embodiment, radiofrequency coil 36 can also surround, be positioned near or integrated within one or more of non-magnetic domains 34. The advantage of positioning radiofrequency coil 36 in closed proximity to non-magnetic domain 34 is to separate radiofrequency coil 36 from domains 22, thereby minimizing interaction between the static magnetic field generated collectively by domain 22 the radiofrequency magnetic fields generated by radiofrequency coil 36. A known phenomenon in NMR is that interactions between magnetic materials and the radiofrequency magnetic field generate the so-called effect of “magnetic acoustic ringing”, in which domains 22 starts to vibrate when the frequency of the radiofrequency magnetic field is close to the resonance frequency of the magnetic structure. In addition, radiofrequency antennas may be excessively loaded once such interactions occur. Thus, non-magnetic domain 34 is preferably constructed and designed for minimizing the load on the radiofrequency antenna and for minimizing magnetic acoustic ringing, e.g., by separating radiofrequency coil 36 from domains 22.

According to a preferred embodiment of the present invention radiofrequency coil 36 may comprises soft magnetic material (such as, but not limited to, soft ferrite, silicon steal and mumrtall) to facilitate the covering of magnetic structure 20 by radiofrequency coil 36.

A particular advantage of magnetic structure 20 is the gradient of the static magnetic field, which allows to use magnetic energy stored therein more efficiently compared to conventional homogenous magnets. In addition, the intrinsic presence of gradient allows generating gradients which are larger than those generated by gradient coils, thereby reduces the need of incorporating large number of gradient coils. For example, if a gradient is to be applied (e.g., for the purpose of MRI slicing) in one direction, no additional gradient coils are to be included, if gradient is to be applied in two directions only one additional gradient coil is included.

Hence, according to a preferred embodiment of the present invention magnetic structure 20 further comprises at least one gradient coil 38. Gradient coils 38 may be positioned in one or two layers, for example an X gradient coil in one layer which is covered by a Y gradient coil in a second layer, where a gradient in Z direction is already provided by magnetic structure 20. Preferably, radiofrequency coil 36 and gradient coil(s) 38 are positioned so as to avoid electromagnetic interactions therebetween. For example, radiofrequency coil 36 may cover one portion of magnetic structure 20 while gradient coil(s) 38 cover another portion of magnetic structure 20. In the embodiments in which magnetic structure comprises non-magnetic domain(s) 34 gradient coil(s) 38 may also be positioned near or integrated within one or more of these domains.

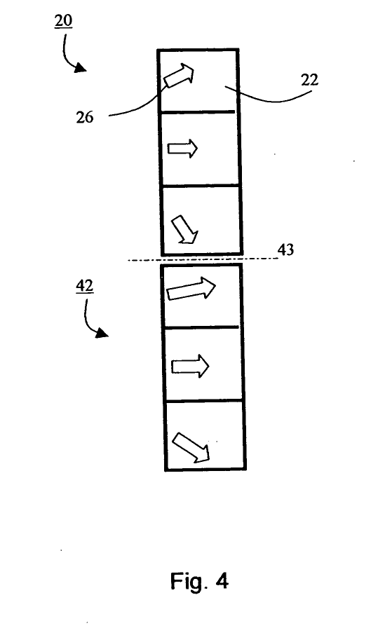

With reference to FIG. 4, magnetic structure 20 may also be combined with at least one additional magnetic structure 42 which also generates a monotonic magnetic field. Magnetic structure 20 may be connected to magnetic structure 42, either firmly or via a device which allow for a relative motion between the two magnetic structures (e.g. a hinge or a pivot). For example, additional magnetic structure 42 may rotate about a transverse axis 43 by one of a plurality of predetermined angles, so as to provide further control on the static magnetic field and its gradient. Alternatively or additionally, the motion of additional magnetic structure 42 with respect to magnetic structure 20 may have a linear nature (e.g., sliding).

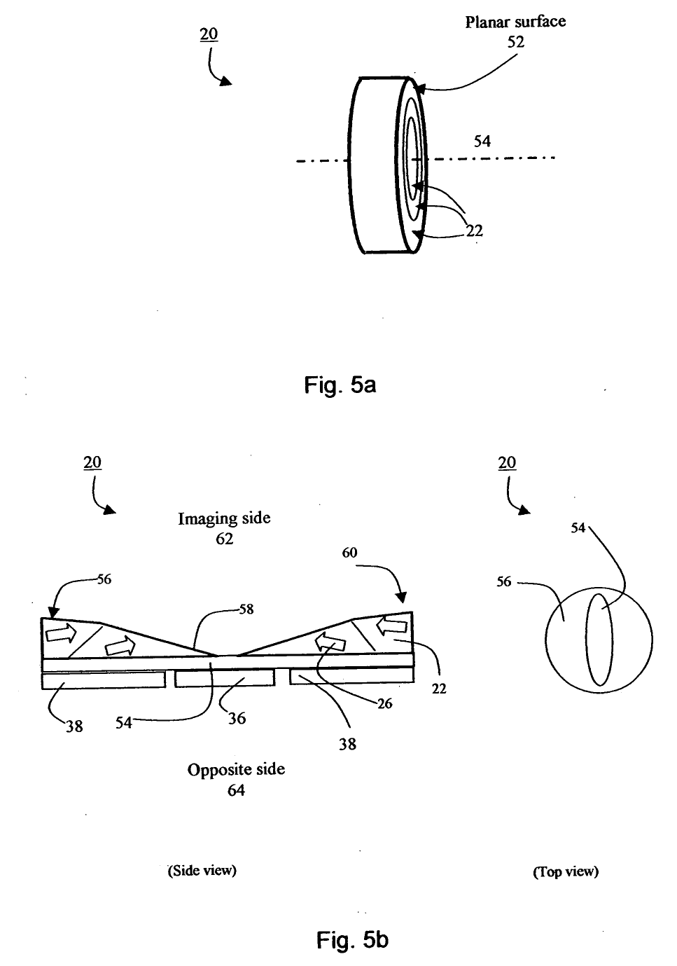

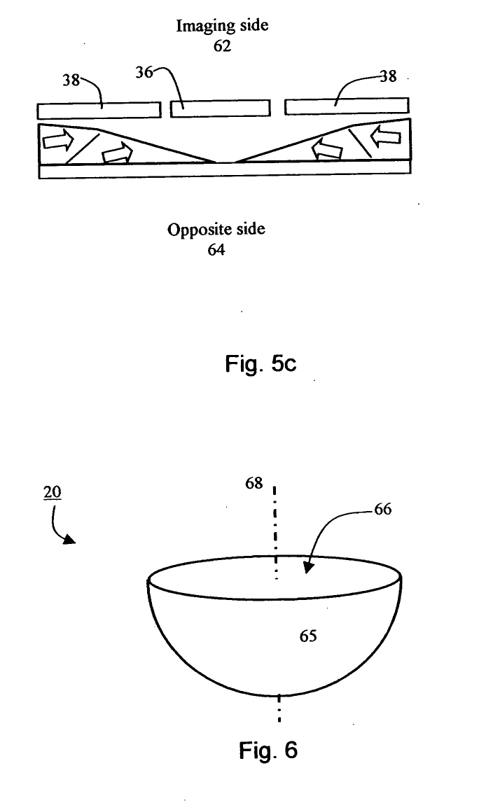

Reference is now made to FIGS. 5a-c, which is a schematic illustration of magnetic structure 20 in which the geometry of volume 24 is characterized by at least one substantially planar surface 52, according to a preferred embodiment of the present invention. Planar surface 52 is defined by an axis 54 which is perpendicular thereto. Such configuration may optionally be used, for example, when magnetic structure 20 is used for imaging and/or analyzing an object whose volume-of-interest is substantially flat, e.g., for surface imaging. In this embodiment, magnetic structure may be shaped, for example as a disk, an ellipse or any other geometrical object having at least one planar surface.

According to a preferred embodiment of the present invention, in the case in which magnetic structure 20 includes planar surface 52, domains 22 are concentrically arranged about a center of magnetic structure 20. The direction of each one of magnetization vectors 26 is preferably selected in accordance with the application in which magnetic structure 20 is used, as further detailed hereinbelow.

Hence, in one embodiment each one of magnetization vectors 26 has a component directed along axis 54, so that the magnetic field, B, is also directed along axis 54. According to a preferred embodiment of the present invention magnetic structure 20 may be assembled from more than one magnetic part, preferably such that these parts are designed to be detachable from one another, so that the operator can optionally and preferably reassemble magnetic structure 20 from different combinations of magnetic parts. For example, if magnetic structure 20 is shaped as a disk where domains 22 are concentric rings, each one of domains 22 may be detachable and connectable to magnetic structure 20. Thus, different ring sizes and/or different magnetization vectors for each ring may be used so as to vary the magnetic characteristics of magnetic structure according to the desired values for a particular measurement.

With reference to FIG. 5b, in another the geometry of volume 24 may also have, in addition to planar surface 54, a non planar surface 56 having a first open end 58, adjacent to planar surface 54, and a second open end 60, far from planar surface 54. As shown in FIG. 5b, the area of first open end 58 is smaller than the area of second open end 60. Such a shape of magnetic structure 20 can be used when the desired direction of the magnetic field, B, is parallel to planar surface 54. In this embodiment a preferred configuration is that domains 22 are arranged within non-planar surface 56, where planar surface 54 may be made of a ferromagnetic material. To provide magnetic field, B, which is parallel to planar surface 54, each one of magnetization vectors 26 preferably has a component directed substantially parallel to planar surface 54. One of ordinarily skill in the art would appreciate that in such configuration magnetic structure 20 may be used for imaging where an imaging side 62 is defined near open end 60 of non-planar surface 56, and an opposite side 64 is defined near planar surface 54.

As stated, magnetic structure 20 may also comprise one or more radiofrequency coils 36 and/or gradient coils 38, which may be located on a particular side of magnetic structure 20. Specifically, radiofrequency coil(s) 36 and/or gradient coil(s) 38 may be located either on the opposite side 64 (FIG. 5b) or on the imaging side 62 (FIG. 5c) of magnetic structure 20.

Reference is now made to FIG. 6, which is a schematic illustration of magnetic structure 20 in which the geometry of volume 24 is a shell 65 having a cavity 66 and a symmetry axis 68, according to a preferred embodiment of the present invention. This embodiment is useful, inter alia in applications in which the analyzed/imaged object can be isolated and inserted into cavity 66 within the shell. For example, this embodiment may be used for performing on-line biopsy. More specifically, a magnetic resonance analysis device may be located in an operating room to be used while the subject is being operated. Thus, during surgery, the physician separates a suspected tissue from the subject and analyzes it in by inserting the tissue into cavity 66 of magnetic structure 20, hence identifies the tissue in real-time without leaving the operating room.

According to the presently preferred embodiment of the invention each of magnetization vectors 26 has a component directed such that the resulting magnetic field, B, is directed along symmetry axis 68. In another embodiment each of magnetization vectors 26 has a component directed such that the magnetic field, B, is directed perpendicularly to symmetry axis 68.

According to a preferred embodiment of the present invention the shell may have any geometry which is suitable for inserting the object to be analyzed into cavity 66. Such geometry includes but is not limited to a hemisphere, a portion of a sphere, an ellipsoid, a portion of ellipsoid, a hyperboloid, a portion of a hyperboloid, a paraboloid, a portion of a paraboloid, a cylinder or a portion of a cylinder. It would be appreciated, however, that the shell shape magnetic structure is also capable of providing a non-homogeneous magnetic field outside cavity 66, preferably close to the open end of cavity 66.

The present invention successfully provides an apparatus for magnetic resonance analysis, generally referred to herein as apparatus 70, which employs the above principles of non-homogenous magnetic field having a gradient. Apparatus 70 may be used either as an imaging (e.g., MRI) or as a non-imaging (e.g., spectroscopy) apparatus for the purpose of analysis of an object by means of magnetic resonance, i.e., by exciting nuclei using a static non-homogenous magnetic field and a radiofrequency magnetic field. The apparatus may be of various sizes, depending on the application for which it is designed. Specifically, apparatus 70 may be used for non-invasive imaging/analyzing medical applications of a portion of an animal or for invasive imaging/analyzing medical applications (e.g., endoscopy, laparoscopy or by miniature capsule).

Before providing a further detailed description of apparatus 70, as delineated hereinabove and in accordance with the present invention, attention will be given to the potential applications and advantages offered thereby.

In magnetic resonance imaging/analysis employing significant gradients, data can be obtained by using pulse sequences which are different than those conventionally used when homogenous static magnetic fields are employed. Such pulse sequences are known in the art and are found, e.g., in P. J. McDonald and B. Newling, “Stray field magnetic resonance imaging”, Rep. Prog. Phys. 61 (1998) 1441-1493. The decay constant of the NMR echoes amplitudes is proportional to the square of the gradient and to the diffusion constant of the analyzed material. Hence, for non-homogenous magnetic fields, broadband radiofrequency field can be used with pulse sequences which are preferably diffusion weighted. In addition, in these systems, the view is in the direction of the gradient thereby avoiding slice selection procedures. As the diffusion constant substantially varies between different materials (or different tissues) the pulse sequences are preferably selected in accordance with the properties of the material which is to be analyzed according the magnetic resonance imaging/analysis.

Another particular advantage of using a static magnetic field having a gradient is that a small change in the field does not change the performance of apparatus 70. Thus, unlike MRI systems with large and homogenous magnets, where the calibration and fine-tuning of the system is a complicate procedure, the calibration procedure of apparatus 70 is simple due to the low sensitivity to small change in the field. Moreover, the low sensitivity permits the apparatus (or the apparatus probe, as further detailed hereinafter) to be relocated, thereby supporting the performance of a different local measurement, and after which the apparatus may subsequently be returned to the original location, for example, for the purpose of validating the preceding measurement.

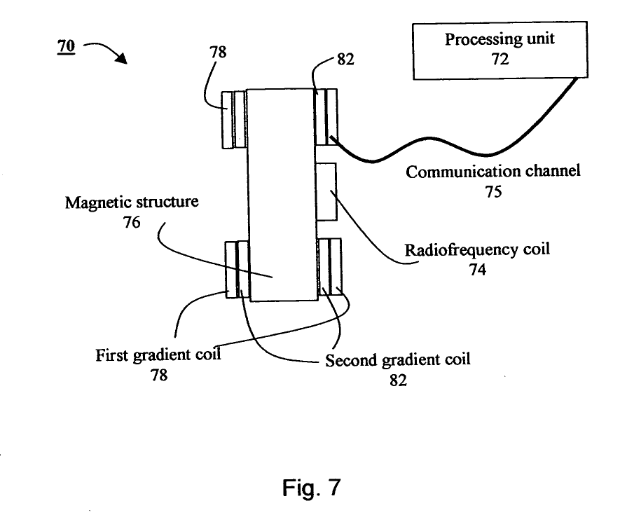

Reference is now made to FIG. 7, which is a schematic illustration of apparatus 70 which comprises a processing unit 72, a radiofrequency coil 74 designed and configured for generating a broadband radiofrequency magnetic field and a magnetic structure 76 for generating a monotonic static magnetic field. Magnetic structure 76 may be any magnetic structure capable of providing a monotonic magnetic field having a gradient, such as, but not limited to, magnetic structure 10. Radiofrequency coil 74 is preferably positioned on magnetic structure 76 as further detailed hereinabove with respect to magnetic structure 10.

Processing unit is communicating with radiofrequency coil 74 via communication channel 75 which may comprise any known communication channel, including, but not limited to, a communication cable or wireless communication (e.g., a transmitter/receiver system). As further detailed hereinunder, wireless communication is typically employed when apparatus 70 (or the magnetic probe thereof) is compact. It is to be understood that although communication channel 75 is graphically represented in FIG. 7 as a physical line, the optional wireless realization of communication channel 75 is not excluded from the scope of the present invention.

According to a preferred embodiment of the present invention apparatus 70 further comprises a first gradient coil 78. In another embodiment, apparatus 70 further comprises a second gradient coil 82. First 78 and second 82 gradient coils each independently serves for generating a magnetic field having a gradient substantially in a direction which is perpendicular to the direction of the gradient of the magnetic field generated by magnetic structure 76. Preferably, first 78 and/or second 82 gradient coils are positioned on the surface of magnetic structure 76, as further detailed hereinabove. However, other configurations in which one or both of the gradient coils is positioned on a remote location are not excluded from the scope of the present invention.

Magnetic structure 76 is preferably manufactured detachable from apparatus 70, for example, for the purpose of replacing it with a different magnetic structure, e.g., having different magnetic characteristics. It is to be understood, however, that in the embodiments in which radiofrequency coil 74, first gradient coil 78 and/or second gradient coil 82 are positioned on the surface of magnetic structure 76, the complete probe of magnetic structure 76 and all the coils is replaceable. Alternatively, the coils (radiofrequency and/or gradient) may be also detachable from magnetic structure 76.

The advantage of the embodiments in which apparatus 70 comprises a replaceable probe is the ability to perform sequential measurements where in each measurement different magnetic field characteristics (e.g. gradient, intensity, radiofrequency) are used. A skilled artisan would appreciate that by varying the magnetic field characteristics one can analyze materials located at different depths in the analyzed object. In addition, it is recognized that different materials are sensitive to different magnetic field characteristics, hence, the use of replaceable magnetic probe facilitate a better analysis of the objects.

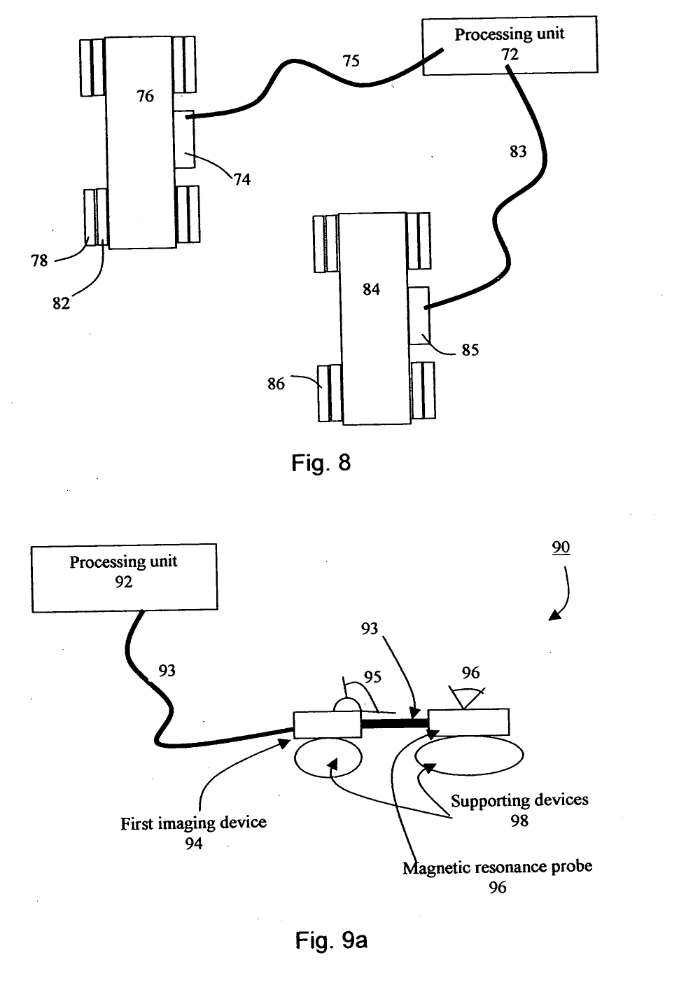

Referring now to FIG. 8, apparatus 70 may optionally comprise more than one magnetic probe having a magnetic structure and a radiofrequency coil and, optionally, one or two gradient coils. Hence, according to a preferred embodiment of the present invention, apparatus 70 preferably comprises at least one additional magnetic structure 84 and at least one additional radiofrequency coil 85, communicating with processing unit 72 via at least one additional communication channel 83. In another embodiment, additional radiofrequency coil 85 and at least one additional magnetic structure 84 are also supplemented by at least one additional gradient coil 86.

Additional radiofrequency coil 85 generates a broad-band radiofrequency magnetic field, additional magnetic structure 84 generates a monotonic static magnetic field and additional gradient coil(s) 86 generate gradients perpendicular to the gradient of the field generated by additional structure 84, as further detailed herein above. Also as detailed hereinabove, additional radiofrequency coil 85 and additional gradient coil 86 may be in proximity to additional magnetic structure 84, or alternatively, additional gradient coil 86 may be kept apart from additional magnetic structure 84.

The embodiments in which apparatus 70 comprises more than one magnetic probe has the advantages that different magnetic probes can be used simultaneously on more then one object or sequentially on the same object, thus further facilitating the analysis of different materials or materials which located at different depths.

According to a preferred embodiment of the present invention, if more than one object is analyzed simultaneously, the objects are sufficiently spaced apart while being analyzed so as to minimize interference effects between the magnetic probes.

The size and shape of the probe is preferably selected in accordance with the application for which it is designed. Thus, in one embodiment, the size and shape of the probe is such that apparatus 70 is suitable for performing an external, non-invasive, measurement. For example, in this embodiment magnetic structure 76 may be shaped so as to allow surface imaging, as further detailed hereinabove, with reference to FIG. 5.

In another embodiment, the size and shape of the probe is such that apparatus 70 is suitable for performing an internal (e.g., endoscopic, laparoscopic) measurement. For example, in this embodiment, magnetic structure 76 may be elongated, as further detailed hereinabove with reference to FIGS. 3-4.

In an additional embodiment, the probe is sufficiently compact so as to allow the probe to be integrated within a capsule which is to be swallowed by a subject. In this embodiment, apparatus 70 further comprise a wireless transmitter which transmits 25 information from radiofrequency cit 74 to processing unit 72.

According to yet another aspect of the present invention there is provided a system for analyzing an object, generally referred to herein as system 90.

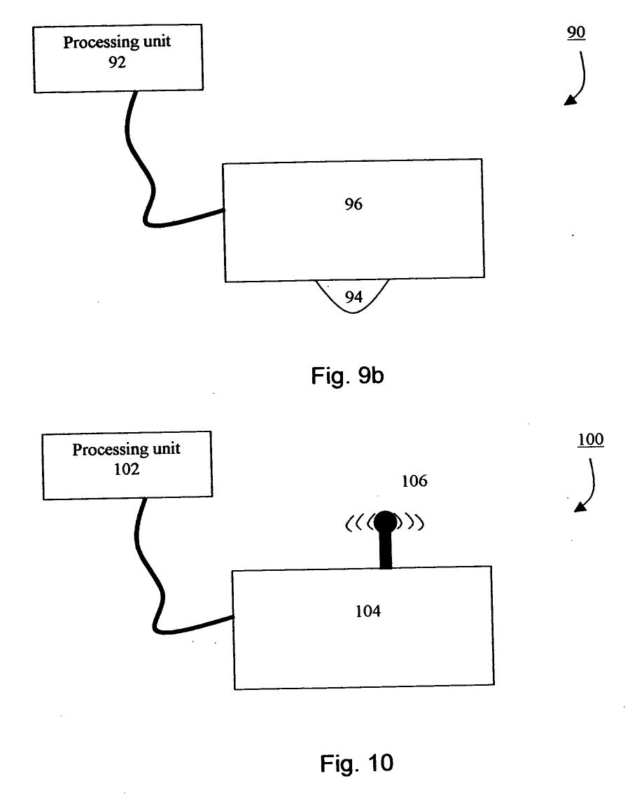

Reference is now made to FIG. 9a-b, which is a schematic illustration of system 90, which comprises a processing unit 92, a first imaging device 94 and a magnetic resonance probe 96. The principles and operations of magnetic resonance probe 96 are similar to the principles and operations of the magnetic probe of apparatus 70 as further detailed herein. System 90 may be used either for invasive procedures (e.g., endoscopy or laparoscopy), as shown in FIG. 9a, or non-invasive procedure, as shown in FIG. 9b. The filed-of-view of device 94 and probe 96 are designated by numerals 95 and 97 respectively.

According to a preferred embodiment of the present invention, first imaging device 94 may optionally be any imaging device, such as, but not limited to, a camera, an ultra-sonic (US) imaging device or a nuclear medicine device sensitive to radioactive radiation.

The position and field-of-view 95 of device 94 is preferably selected so that both probe 96 and device 94 operate simultaneously. According to a preferred embodiment of the present invention both probe 96 and device 94 may be supported by supporting devices 98 (e.g., a supporting balloon). Supporting devices 98 are typically used in endoscopic procedures where the endoscope is to be fixed at a particular location.

For an endoscopic system, probe 96 is preferably located at the distal end of the endoscope while device 94 is located behind probe 96. In this embodiment the field of view of device 94 overlaps a portion of the field of view of probe 96. If, for example, device 94 is a camera, device 94 can be so positioned so that probe 96 is within the field of view of the camera (device 94). Thus, the camera (device 94) is used for navigating the endoscope and for identifying the region which is to be analyzed by probe 96. In the embodiment in which device 94 is an US imaging device, device 94 is used for identifying tissues structures and geometries whereas probe 96 is used for further analysis. In the embodiment in which device 94 is a nuclear medicine device, device 94 may be used for the purpose of identifying tumors via radiation. Specifically, prior to the identification procedure, a radioactive material is administrated to a subject. As, the concentration of the radioactive material in tumors is larger than the concentration of the radioactive material in surrounding region, the nuclear medicine device (device 94) identifies regions of high radioactivity as tumors. Nuclear medicine devices suitable for endoscopy are known in the art and are manufactured and distributed, e g., by Izmel project Israel.

According to a preferred embodiment of the present invention system 90 may include more than one imaging device and one probe; for example, a combination of a camera, an US device and probe 96 may be used, where the camera is used for navigation, the US device for geometrical identification and probe 96 for magnetic resonance analysis/imaging.

Device(s) 94 and probe(s) 96 may by either interconnected via a communication cable 93, as shown in FIG. 9a, or alternatively, device(s) 94 may be mounted on probe(s) 96, as shown in FIG. 9b. Preferably, communication cable 93 is used to connect device(s) 94 and probe(s) 96 in the embodiment in which system 90 is used for invasive procedures. In this embodiment, communication cable 93 may be either flexible (e.g., during navigation) or fixed (e.g., while imaging).

Reference is now made to FIG. 10, which is a schematic illustration of a system for analyzing an object, according to yet an additional aspect of the present invention.

In this embodiment, the system, generally referred to herein as system 100, comprises a processing unit 102, a magnetic resonance probe 104 and a position tracking system 106. The principles and operations of magnetic resonance probe 104 are similar to the principles and operations of apparatus 70 as further detailed hereinabove.

Position tracking system 106 serves for determining a position probe 104. The advantage of position tracking system 106 is twofold. First, as stated, it may be desired to perform more than one measurement at the same location using different magnetic characteristics, and/or for performing some therapeutic treatment (e.g., by radiation). Hence, position tracking system 106 is used for the purpose of relocating probe 104 at the location in which a previous measurement had been performed. Second, it may be desired to perform a plurality of measurements at a plurality of locations thereby to map a substantially large region of interest. In this case, position tracking system 106 is used for the purpose of reconstruction of the complete map of the region of interest once all the measurements are accomplished. Specifically, position tracking system 106 stores the location of each measurement in real-time while in the processing stage, the data are organized according to the respective location.

Position tracking systems per se are well known in the art and may use any one of a plurality of approaches for the determination of position in a two- or three-dimensional space as is defined by a system-of-coordinates of a plurality of degrees-of-freedom. Some position tracking systems employ movable physical connections and appropriate, movement monitoring devices (e.g., potentiometers) to keep track of position changes. Thus, such systems, once zeroed, keep track of position changes to thereby determine actual positions at all times. One example for such a position tracking system is an articulated arm, which can be connected to probe 104.

An articulated arm includes n arm members and a base, which can therefore provide positional data in n degrees-of-freedom. Monitoring positional changes may be effected in any one of several different ways. For example, providing each arm member with, e.g., potentiometers or optical encoders used to monitor the angle between adjacent arm members, to thereby monitor the angular change of each such arm member with respect to adjacent arm members. The angular change allows determining the position in space of probe 104, which is physically connected to the articulated arm.

Another example of a position tracking system is an assortment of three triaxially (e.g., co-orthogonally) oriented accelerometers which may be used to monitor the positional changes of probe 104 with respect to a space.

Still another position tracking system employs an array of receivers/transmitters which are spread in known positions in a three-dimensional space and, in addition, one or more transmitters/receivers, which are in physical connection with probe 104. Time based triangulation and/or phase shift triangulation are used in such cases to periodically determine the position of probe 104. Examples of such a position tracking systems employed in a variety of contexts using acoustic (e.g., ultrasound) electromagnetic radiation (e.g., infrared, radiofrequency) or magnetic field and optical decoding are disclosed in, for example, U.S. Pat. Nos. 5,412,619; 6,083,170; 6,063,022; 5,954,665; 5,840,025; 5,718,241; 5,713,946; 5,694,945; 5,568,809; 5,546,951; 5,480,422 and 5,391,199, which are incorporated by reference as if fully set forth herein.

It is to be understood, however, that although position tracking system 106 has been describe in connection to system 100, it is not intended to limit the scope of the present invention. Hence, other combinations with position tracking system 106 are within the scope of the present invention. Specifically, position tracking system 106 may be combined with apparatus 70 and/or with system 90.

All the above apparatuses and systems, and any other device, apparatus and/or system which employ the above magnetic structures may be employed on many objects which hare to be imaged and/or analyzed. These include, but are not limited to, an animal (e.g., a human being), an organ of an animals a tissue, a swollen elastomer and a food material. In addition, the object may liquid (e.g., oil) or molecules which are present (e.g., dissolved, dispersed or emulsed) in a solvent.

Additionally, the above apparatuses and systems, and any other device, apparatus and/or system which employ the above magnetic structures are preferably designed so as to distinguish between different types of cells and/or different types of substrates. This can be done, for example, by manufacturing the magnetic structure and the radiofrequency coil such that the monotonic static magnetic field and the radiofrequency magnetic field generate predetermined and different magnetic resonance responses in predetermined and different types of cells and/or different types of substrates. Examples of different types of cells include, but are not limited to, a malignant tumor and a benign tumor, or tissue from different parts of the body, as further detailed hereinabove. Different types of substrates may include a first substance and at least one additional substance, which is present in or surrounded by the first substance. For example, water in concrete or impurities in oil.

Still additionally, the above apparatuses and systems, and any other device, apparatus and/or system which employ the above magnetic structures is preferably designed so as to monitor changes in the state of the analyzed material or substrate, which are realized by dynamically (time-dependent) resonance characteristics. This can be done, for example, by manufacturing the magnetic structure and the radiofrequency coil such that the monotonic static magnetic field and the radiofrequency magnetic field generate a magnetic resonance response in substances having dynamical resonance characteristics.

Additional objects, advantages and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which, together with the above descriptions, illustrate the invention in a non limiting fashion.

### Example 1

### A Variation of the Functional Under a Set of Constraints

In the following example, the desired characteristic of the magnetic field is obtained by minimizing the functional U₀ of Equation 3 using a set of N constraints, which are categorized into two types.

A first type of constraint includes N₁ equality constraints in which there are N_{J} components of the query magnetic field, Bᵢ^{m}, which are predetermined:

$${B}_{i}^{m} ( {x}_{m} ) = {\int }_{D}^{} ⅆ {V}^{′} {G}_{i , k} ( {x}^{′} , {x}_{m} ) {J}_{k} ( {x}^{′} ) , \qquad ( EQ . 5 )$$

where i=1, . . . , N₁, D is the second geometry and G is the green function defined in Equation 1 above.

A second type of constraint includes N-N₁ inequality constraints bounding the derivative of one B component. This type is further subdivided into N-N₂ constraints bounding the derivative of one B component from below, denoted herein by g^{m}^{_{d}}, and N₂-N₁ constraints bounding the derivative of one B component from above, denoted herein by g^{m}^{ᵤ}

$${g}_{i , k}^{{m}_{u}} ( {x}_{m} ) \geq \frac{{\hat{x}}_{i} \partial }{\partial {x}_{i}} {\int }_{D}^{} ⅆ {V}^{′} {G}_{k , l} ( {x}^{′} , {x}_{m} ) {J}_{l} ( {x}^{′} ) \geq {g}_{i , k}^{{m}_{d}} ( {x}_{m} ) . \qquad ( EQ . 6 )$$

An additional constraint in this example is that the norm of the remanence distribution is constant (|J(x)|=J₀) over the second geometry, D, which can be written mathematically as a requirement that the gradient of the norm square vanishes:
∇(J(x)·J(x))=0. (EQ. 7)

The extremum problem under the above constraints is solved by the method of Lagrange multipliers. Specifically, a new functional, U₁, is constructed according to the following equation:

$${U}_{1} ( J , λ , α ) = {U}_{0} + {\sum }_{n = 1}^{{N}_{1}} {λ}_{n} [ {B}_{i}^{n} - {\int }_{D}^{} ⅆ {V}^{′} {G}_{i , j} ( {x}^{′} , {x}_{n} ) {J}_{j} ( {x}^{′} ) ] + {\sum }_{n = {N}_{1}}^{n = {N}_{2}} {λ}_{n}^{u} [ {g}_{i , k}^{{n}_{u}} - \frac{{\hat{x}}_{k} \partial }{\partial {x}_{k}} {\int }_{D}^{} {G}_{i , l} ( {x}^{′} , {x}_{n} ) {J}_{l} ( {x}^{′} ) ] + {\sum }_{n = {N}_{2}}^{n = N} {λ}_{n}^{d} [ {g}_{i , k}^{{n}_{d}} - \frac{{\hat{x}}_{k} \partial }{\partial {x}_{k}} {\int }_{D}^{} {G}_{i , l} ( {x}^{′} , {x}_{n} ) {J}_{l} ( {x}^{′} ) ] - \frac{1}{2} {\int }_{d}^{} ⅆ {V}^{′} α ( x ) \cdot ∇ [ J ( x ) \cdot J ( x ) ] , \qquad ( EQ . 8 )$$

where λₙ, n=1, . . . , N are scalar constant Lagrange multipliers and α(x) is a vector function Lagrange multiplier. The extremum problem has a solution if the following Kuhn-Tucker conditions are satisfied:

$$\begin{matrix}{λ}_{n} \geq 0 for n = {N}_{1} + 1 , … , N ; & ( EQ . 9 ) \\ and & \\ {g}_{i , k}^{{n}_{u}} ( {x}_{m} ) \geq \frac{{\hat{x}}_{i} \partial }{\partial {x}_{i}} {\int }_{D}^{} ⅆ {V}^{′} {G}_{k , l} ( {x}^{′} , {x}_{m} ) {J}_{l}^{*} ( {x}^{′} ) \leq {g}_{i , k}^{{n}_{d}} ( {x}_{m} ) , & ( EQ . 10 )\end{matrix}$$

where, J*(x) is the function J(x) at the extremum

The minimum of the functional U₀, given the above constraint and condition, is obtained by vanishing the variation of the new functional U₁:

$$\begin{matrix}\frac{δ {U}_{1}}{δ J ( x )} = 0 ; & ( EQ . 11 ) \\ \frac{δ {U}_{1}}{δ {λ}_{m} ( x )} = 0 for m = 1 , 2 , … {N}_{1} ; & ( EQ . 12 ) \\ \frac{δ {U}_{1}}{δ {λ}_{m} ( x )} = 0 or {λ}_{m} = 0 , for m = {N}_{1} + 1 … N ; & ( EQ . 13 ) \\ and & \\ \frac{δ {U}_{1}}{δ α ( x )} = 0. & ( EQ . 14 )\end{matrix}$$

It is convenient to define the following notations for the Green's function and its derivatives:

$$\begin{matrix}{g}_{n} ( x ) = {G}_{k , j} ( {x}^{′} , {x}_{n} ) for n = 1 , 2 , … , {N}_{1} ; & ( EQ . 15 ) \\ and & \\ {g}_{n} ( x ) = \frac{{\hat{x}}_{i} \partial }{\partial {x}_{i}} {G}_{k , j} ( {x}^{′} , {x}_{n} ) for n = {N}_{1} + 1 , … , N . & ( EQ . 16 )\end{matrix}$$

Using the definitions of Equations 15 and 16, Equation 13 becomes:

$$\begin{matrix}[ 1 + ∇ \cdot α ( x ) ] {J}_{k} ( x ) = {\sum }_{n = 1}^{N} {λ}_{n} {g}_{n} ( x ) , & ( EQ . 17 ) \\ thus , solving for J ( x ) one finds & \\ J ( x ) = {J}_{0} \frac{{\sum }_{n = 1}^{N} {λ}_{n} {g}_{n} ( x )}{\vert {\sum }_{n = 1}^{N} {λ}_{n} {g}_{n} ( x ) \vert} . & ( EQ . 18 )\end{matrix}$$

For a given J₀ the set {λₙ} of Lagrange scalar constant multipliers can be found by solving the following set of equations:

$$\begin{matrix}{B}_{i}^{m} ( {x}_{m} ) = {J}_{0} {\int }_{D}^{} ⅆ {V}^{′} {G}_{i , k} ( {x}^{′} , {x}_{m} ) \frac{{\sum }_{n = 1}^{N} {λ}_{n} {g}_{n} ( x )}{\vert {\sum }_{n = 1}^{N} {λ}_{n} {g}_{n} ( x ) \vert} \\ and \\ {g}_{i , k}^{{m}_{d}} ( {x}_{m} ) = \frac{{\hat{x}}_{i} \partial }{\partial {x}_{i}} {\int }_{D}^{} ⅆ {V}^{′} {G}_{k , l} ( {x}^{′} , {x}_{m} ) \frac{{\sum }_{n = 1}^{N} {λ}_{n} {g}_{n} ( x )}{\vert {\sum }_{n = 1}^{N} {λ}_{n} {g}_{n} ( x ) \vert} \\ for {λ}_{m}^{u} = 0 and {λ}_{m}^{d} \neq 0 \\ {g}_{i , k}^{{m}_{u}} ( {x}_{m} ) = \frac{{\hat{x}}_{i} \partial }{\partial {x}_{i}} {\int }_{D}^{} ⅆ {V}^{′} {G}_{k , l} ( {x}^{′} , {x}_{m} ) \frac{{\sum }_{n = 1}^{N} {λ}_{n} {g}_{n} ( x )}{\vert {\sum }_{n = 1}^{N} {λ}_{n} {g}_{n} ( x ) \vert} \\ for {λ}_{m}^{u} \neq 0 and {λ}_{m}^{d} = 0.\end{matrix} \qquad ( EQ . 19 )$$

In some cases the obtained solution already satisfied the N-N₁ constraints of Equation 6. In this case the constant Lagrange multipliers λₘ^{u} and λₘ^{d} are set to zero in the functional of Equation 8.

Hence, once the set {λₙ} are known, the remanence distribution is given by Equation 18 above.

### Example 2

### Cylindrical Magnets

Two cylindrical magnets were designed according to the method of the present invention. The functional and constraints were as in Example 1. A first cylindrical magnet was designed so as to generate a radial magnetic field and a second cylindrical magnet was designed so as to generate an axial magnetic field. The dimensions of both cylinders were 1.5 cm in radius and 5 cm in height, the total magnetization was 1.4 T and the magnets were designed to include 3 domains.

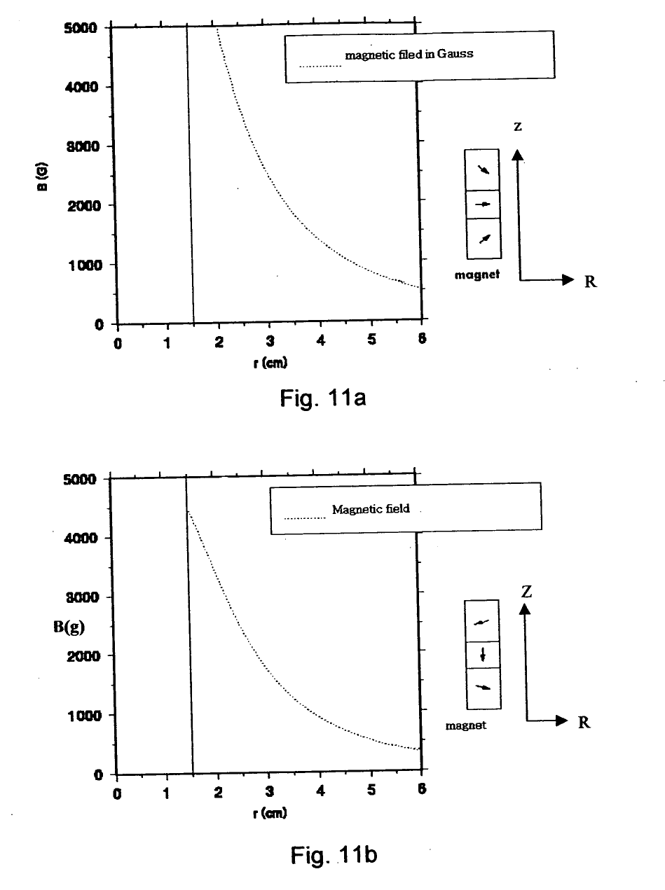

FIGS. 11a-b show, respectively for the first and second cylindrical magnets, the magnetizations of each domain and the magnetic field, B, as a function of the distance, r, from the center of each cylinder. A substantially rapid drop of the magnetic field is seen, from about 4500 Gausses near the surface of the magnets to less than 1000 Gauss at r=6 cm.

### Example 3

### A Surface Magnet

A surface magnet was designed according to the method of the present invention. The functional and constraints were as in Example 1. The surface magnet was designed as a disk so as to generate an axial magnetic field. The dimensions of the disk were 6 cm in radius and 5 cm in height, the total magnetization was 1.4 T and the magnet was designed to include 3 concentric domains.

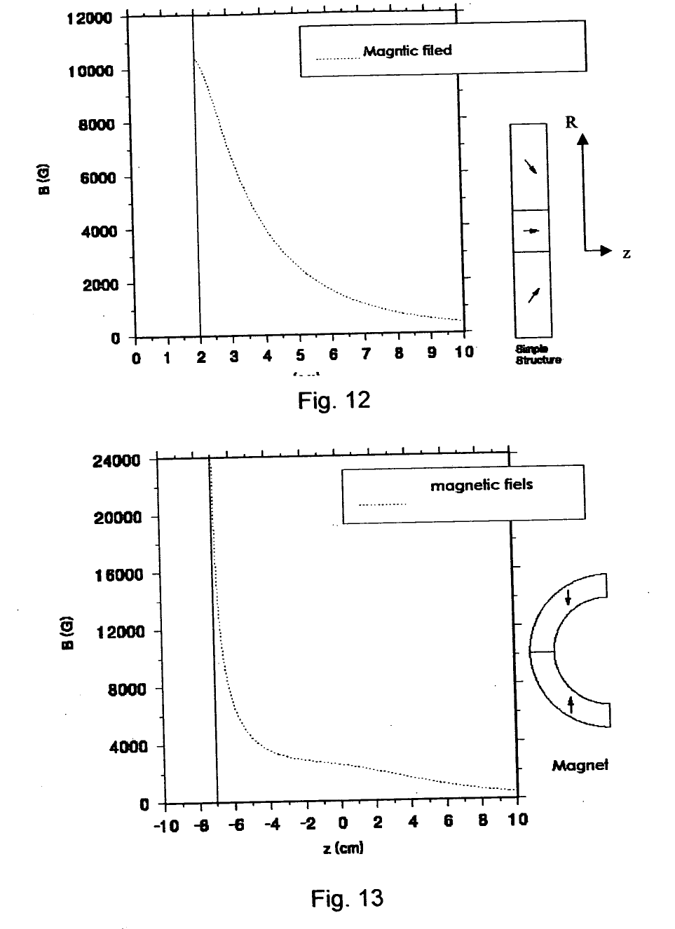

FIG. 12 shows the magnetizations of each domain and the magnetic field, B, as a function of the distance, z, from the surface of the disk. A substantially rapid drop of the magnetic field is seen, from about 10000 Gausses near the surface of the magnet to less than 1000 Gauss at z=10 cm.

### Example 4

### A Shell Magnet

A shell magnet was designed according to the method of the present invention. The functional and constraints were as in Example 1. The shell magnet was designed as a hemisphere so as to generate an axial magnetic field. The dimensions of the hemisphere were 10 cm in external radius and 7 cm in internal radius, the total magnetization was 1.4 T and the magnet was designed to include 2 domains, symmetrically positioned a long a symmetry axis. FIG. 13 shows the magnetizations of each domain and the magnetic field, B, as a function of the distance, z, from the surface of the hemisphere. It can be seen the magnetic field intensity is larger for z<0, i.e., within the hemisphere.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition citation or identification of any reference in this application shall not be construed as an admission that such reference is available as prior art to the present invention.

What is claimed is:

1. A method of designing a magnetic structure for providing a monotonic static magnetic field for magnetic resonance analysis, the method comprising:
selecting a first geometry defining a volume-of-interest;
selecting a magnetic field query, defined on a plurality of coordinates within said first geometry, said magnetic field query being monotonic;
selecting a second geometry defining the magnetic structure; and
calculating a remanence distribution within said second geometry by using said first geometry, said second geometry and said magnetic field query, thereby designing the magnetic structure.

2. The method of claim 1, further comprising optimizing the monotonic static magnetic field, by repeating said selecting said first and second geometries and said magnetic field query and repeating said calculating of said remanence distribution.

3. The method of claim 1, wherein said first geometry is selected so that a maximal value of at least one component of said magnetic field query is above a predetermined threshold.

4. The method of claim 3, wherein said predetermined threshold is selected so as to optimize a signal-to-noise ratio and a signal to contrast.

5. The method of claim 1, wherein said second geometry is selected so that a maximal value of at least one component of said magnetic field query is above a predetermined threshold.

6. The method of claim 5, wherein said predetermined threshold is selected so as to optimize a signal-to-noise ratio and a signal to contrast.

7. The method of claim 1, wherein said remanence distribution is calculated by constructing a functional of said remanence distribution and minimizing said functional using a set of constraints.

8. The method of claim 1, wherein said remanence distribution is calculated by constructing a functional of said remanence distribution and maximizing said functional using a set of constraints.

9. The method of claim 7, wherein each constraint of said set of constraints is selected from the group consisting of an equality constraint and an inequality constraint.

10. The method of claim 7, wherein said set of constraints are selected so as to optimize the monotonic static magnetic field.

11. A magnetic structure for magnetic resonance analysis, the magnetic structure comprising a plurality of domains, arranged within a volume having predetermined geometry, each of said plurality of domains being characterized by a predetermined and different magnetization vector;
wherein said predetermined geometry and said plurality of domains are selected so as to generate a monotonic static magnetic field having a gradient.

12. The magnetic structure of claim 11, wherein said plurality of domains comprises at least three domains.

13. The magnetic structure of claim 11, wherein said plurality of domains comprises at least four domains.

14. The magnetic structure of claim 11, wherein said predetermined geometry is selected from the group consisting of a cylinder, a disk, a prism, a sphere, a hemisphere, a portion of a sphere, an ellipsoid, a portion of ellipsoid, a hyperboloid, a portion of a hyperboloid, a paraboloid, a portion of a paraboloid, a cylindrical shell, a portion of a cylindrical, shell, a polyhedron shell and a portion of a polyhedron shell.

15. The magnetic structure of claim 11, wherein said predetermined geometry is elongated with respect to a longitudinal axis, and further wherein a size of the magnetic structure is selected so as to allow the magnetic structure to be inserted into a body of a subject by endoscopy.

16. The magnetic structure of claim 15, wherein said plurality of domains are arranged along said longitudinal axis, and further wherein a magnetization vector of each domain has a component directed perpendicular to said longitudinal axis so that said monotonic static magnetic field is also in a direction perpendicular to said longitudinal axis.

17. The magnetic structure of claim 15, wherein said plurality of domains are arranged along said longitudinal axis, and further wherein a magnetization vector of each domain has a component directed parallel to said longitudinal axis so that said monotonic static magnetic field is also in a direction parallel to said longitudinal axis.

18. The magnetic structure of claim 15, further comprising at least one additional magnetic structure designed connectable to the magnetic structure, wherein said at least one additional magnetic structure capable of generating a monotonic static magnetic field.

19. The magnetic structure of claim 18, wherein said at least one additional magnetic structure operable to rotate about a transverse axis by one of a plurality of predetermined angles.

20. The magnetic structure of claim 15, wherein said monotonic static magnetic field varies along said longitudinal axis.

21. The magnetic structure of claim 15, wherein said magnetization vectors vary along a radial direction, said radial direction being perpendicular to said longitudinal axis.

22. The magnetic structure of claim 15, wherein said magnetization vectors vary along an azimuthal direction said azimuthal direction being perpendicular to said longitudinal axis.

23. The magnetic structure of claim 11, further comprising at least one non-magnetic domain located so as to optimize a profile of said monotonic static magnetic field.

24. The magnetic structure of claim 23, designed connectable to a radiofrequency antenna and further wherein said at least one non-magnetic domain is constructed and designed for minimizing a load on said radiofrequency antenna and for minimizing magnetic acoustic ringing.

25. The magnetic structure of claim 23, wherein a size of said at least one non-magnetic domain is selected so as to be surrounded by at least one radiofrequency coil.

26. The magnetic structure of claim 23, wherein a size of said at least one non-magnetic domain is selected so as to minimize an amount of magnetic material present in the magnetic structure.

27. The magnetic structure of claim 23, wherein said at least one radiofrequency coil comprises a soft magnetic material.

28. The magnetic structure of claim 11, wherein said predetermined geometry is characterized by at least one substantially planar surface, said at least one substantially planar surface defined by an axis being perpendicularly to said at least one substantially planar surface.

29. The magnetic structure of claim 28, wherein said, plurality of domains are concentrically arranged about a center of the magnetic structure, and further wherein a magnetization vector of each domain has a component directed along said axis, so that said monotonic static magnetic field is also directed along said axis.

30. The magnetic structure of claim 11, wherein said predetermined geometry is characterized by a planar surface and a non-planar surface, said non-planar surface having a first open end, adjacent to said planar surface, and a second open end, far from said planar surface, where an area of said first open end is smaller than an area of said second open end.

31. The magnetic structure of claim 30, wherein said plurality of domains are arranged within said non-planar surface, and further wherein a magnetization vector of each domain has a component directed substantially parallel to said planar surface, so that said monotonic static magnetic field is also directed substantially parallel to said planar surface.

32. The magnetic structure of claim 11, wherein said predetermined geometry is a shell having a cavity and a symmetry axis, said shell is selected from the group consisting of a hemisphere, a portion of a sphere, an ellipsoid, a portion of ellipsoid, a hyperboloid, a portion of a hyperboloid, a paraboloid, a portion of a paraboloid, a cylindrical shell, a portion of a cylindrical shell, a polyhedron shell and a portion of a polyhedron shell.

33. The magnetic structure of claim 32, wherein a magnetization vector of each domain is directed along said symmetry axis so that said monotonic static magnetic field is also directed along said symmetry axis.

34. The magnetic structure of claim 32, wherein a magnetization vector of each domain is directed perpendicularly to said symmetry axis so that said monotonic static magnetic field is also directed perpendicularly to said symmetry axis.

35. An apparatus for magnetic resonance analysis, the apparatus comprising:
a processing unit;
a radiofrequency coil designed and configured for generating a broad-band radiofrequency magnetic field; and
a magnetic structure for generating a monotonic static magnetic field having a gradient, said magnetic structure comprising a plurality of domains, said plurality of domains being arranged within a volume having predetermined geometry, each of said plurality of domains being characterized by a predetermined and different magnetization vector, wherein said predetermined geometry and said plurality of domains are selected so as to generate said monotonic static magnetic field.

36. The apparatus of claim 35, further comprising a first gradient coil for generating a magnetic field having a gradient substantially in a first transverse direction.

37. The apparatus of claim 36, further comprising a second gradient coil for generating a magnetic field having a gradient substantially in a second transverse direction.

38. The apparatus of claim 36, wherein said first gradient coil is positioned on a surface of said magnetic structure.

39. The apparatus of claim 37, wherein said first and said second gradient coils are positioned on a surface of said magnetic structure.

40. The apparatus of claim 37, wherein said first and said second gradient coils are arranged in one layer.

41. The apparatus of claim 37, wherein said first and said second gradient coils are arranged separate layers.

42. The apparatus of claim 36, wherein said radiofrequency coil is positioned on a surface said magnetic structure and further wherein said first gradient coil and said radiofrequency coil are arranged in one layer.

43. The apparatus of claim 37, wherein said radiofrequency coil is positioned on a surface of said magnetic structure and further wherein said first gradient coil and said radiofrequency coil are arranged in one layer.

44. The apparatus of claim 36, wherein said magnetic structure is detachable.

45. The apparatus of claim 36, wherein said magnetic structure is replaceable.

46. The apparatus of claim 36, wherein said magnetic structure comprises at least two parts, each independently operable to rotate about a longitudinal axis.

47. The apparatus of claim 36, wherein said magnetic structure comprises at least two parts, each independently operable to move along a longitudinal axis.

48. The apparatus of claim 35, wherein said plurality of domains comprises at least three domains.

49. The apparatus of claim 35, wherein said plurality of domains comprises at least four domains.

50. The apparatus of claim 35, wherein said predetermined geometry is selected from the group consisting of a cylinder, a disk, a prism, a sphere, a hemisphere, a portion of a sphere, an ellipsoid, a portion of ellipsoid, a hyperboloid, a portion of a hyperboloid, a paraboloid, a portion of a paraboloid, a cylindrical shell, a portion of a cylindrical shell, a polyhedron shell and a portion of a polyhedron shell.

51. The apparatus of claim 35, wherein said predetermined geometry is elongated with respect to a longitudinal axis, and further wherein a size of said magnetic structure is selected so as to allow said magnetic structure to be inserted into a body of a subject by endoscopy.

52. The apparatus of claim 51, wherein said plurality of domains are arranged along said longitudinal axis, and further where in a magnetization vector of each domain has a component directed perpendicular to said longitudinal axis so that said monotonic static magnetic field is also in a direction perpendicular to said longitudinal axis.

53. The apparatus of claim 51, wherein said plurality of domains are arranged along said longitudinal axis, and further wherein a magnetization vector of each domain has a component directed parallel to said longitudinal axis so that said monotonic static magnetic field is also in a direction parallel to said longitudinal axis.

54. The apparatus of claim 51, further comprising at least one additional magnetic structure designed connectable to said magnetic structure, wherein said at least one additional magnetic structure capable of generating a monotonic static magnetic field.

55. The apparatus of claim 54, wherein said at least one additional magnetic structure operable to rotate about a transverse axis by one of a plurality of predetermined angles.

56. The apparatus of claim 51, wherein said monotonic static magnetic field varies along said longitudinal axis.

57. The apparatus of claim 51, wherein said magnetization vectors vary along a radial direction, said radial direction being perpendicular to said longitudinal axis.

58. The apparatus of claim 51, wherein said magnetization vectors vary along an azimuthal direction, said azimuthal direction being perpendicular to said longitudinal axis.

59. The apparatus of claim 35, said magnetic structure further comprising at least one non-magnetic domain located so as to optimize a profile of said monotonic static magnetic field.

60. The apparatus of claim 59, wherein said radiofrequency coil comprises a radiofrequency antenna and further wherein said at least one non-magnetic domain is constructed and designed for minimizing a load on said radiofrequency antenna and for minimizing magnetic acoustic ringing.

61. The apparatus of claim 59, wherein a size of said at least one non-magnetic domain is selected so as to be surrounded by at least one radiofrequency coil.

62. The apparatus of claim 59, wherein a size of said at least one non-magnetic domain is selected so as to minimize an amount of magnetic material present in said magnetic structure.

63. The apparatus of claim 59, wherein said at least one radiofrequency coil comprises a soft magnetic material.

64. The apparatus of claim 35, wherein said predetermined geometry is characterized by at least one substantially planar surface, said at least one substantially planar surface defined by an axis being perpendicularly to said at least one substantially planar surface.

65. The apparatus of claim 64, wherein said plurality of domains are concentrically arranged about a center of said magnetic structure, and further wherein a magnetization vector of each domain has a component directed along said axis, so that said monotonic static magnetic field is also directed along said axis.

66. The apparatus of claim 35, wherein said predetermined geometry is characterized by a planar surface and a non-planar surface, said non-planar surface having a first open end, adjacent to said planar surface, and a second open end, far from said planar surface, where an area of said first open end is smaller than an area of said second open end.

67. The apparatus of claim 66, wherein said plurality of domains are arranged within said non-planar surface, and further wherein magnetization vector of each domain has a component directed substantially parallel to said planar surface, so that said monotonic static magnetic field is also directed substantially parallel to said planar surface.

68. The apparatus of claim 35, wherein said predetermined geometry is a shell having a cavity and a symmetry axis, said shell is selected from the group consisting of a hemisphere, a portion of a sphere, an ellipsoid, a portion of ellipsoid, a hyperboloid, a portion of a hyperboloid, a paraboloid, a portion of a paraboloid, a cylindrical shell, a portion of a cylindrical shell, a polyhedron shell and a portion of a polyhedron shell.

69. The apparatus of claim 68, wherein a magnetization vector of each domain is directed along said symmetry axis so that said monotonic static magnetic field is also directed along said symmetry axis.

70. The apparatus of claim 68, wherein a magnetization vector of each domain is directed perpendicularly to said symmetry axis so that said monotonic static magnetic field is also directed perpendicularly to said symmetry axis.

71. The apparatus of claim 35, further comprising at least one additional radiofrequency coil for generating a broad-band radiofrequency magnetic field and at least one additional magnetic structure for generating a monotonic static magnetic field having a gradient, wherein each of said at least one additional radiofrequency coil is in proximity to said at least one additional magnetic structure.

72. The apparatus of claim 35, further comprising a power supply and a wireless transmitter for transmitting information from the radiofrequency coil, wherein a size of said radiofrequency coil and a size of said magnetic structure are selected so as to capsule said radiofrequency coil and said magnetic structure into a compact probe to be swallowed by a subject.

73. The apparatus of claim 35, wherein said magnetic structure and said radiofrequency coil are designed and constructed so that said monotonic static magnetic field and said radiofrequency magnetic are capable of generating predetermined and different magnetic resonance responses in predetermined and different types of cells.

74. The apparatus of claim 73, wherein said predetermined types of cells are selected from the group consisting of a part of a tumor, a part of a malignant tumor, a part of a blood vessel tissue, a part of a pathological tissue and a part of a restenotic tissue.

75. The apparatus of claim 35, wherein said magnetic structure and said radiofrequency coil are designed and constructed so that said monotonic static magnetic field and said radiofrequency magnetic field are capable of generating a predetermined and different magnetic resonance responses in a first substance and in at least one additional substance present in or surrounded by said first substance, thereby distinguishing between said first substance and said at least one additional substance.

76. The apparatus of claim 35, wherein said magnetic structure and said radiofrequency coil are designed and constructed so that said monotonic static magnetic field and said radiofrequency magnetic field are capable of generating a magnetic resonance response in at least one substance having dynamical resonance characteristics, said dynamical resonance characteristics being time-dependent.

77. The apparatus of claim 76, design and constructed to monitor said dynamical resonance characteristics.

78. A magnetic structure for magnetic resonance analysis, comprising a structure defined according to a remanence distribution, said remanence distribution being determined according to a first geometry for defining a volume-of-interest, a second geometry for defining the magnetic structure and a magnetic field query being defined on a plurality of coordinates within said first geometry, said magnetic field query being monotonic.

79. The magnetic structure of claim 78, wherein said first geometry is selected so that a maximal value of at least one component of said magnetic field query is above a predetermined threshold.

80. The magnetic structure of claim 79, wherein said predetermined threshold is selected so as to optimize a signal-to-noise ratio and a signal to contrast.

81. The magnetic structure of claim 78, wherein said second geometry is selected so that a maximal value of at least one component of said magnetic field query is above a predetermined threshold.

82. The magnetic structure of claim 81, wherein said predetermined threshold is selected so as to optimize a signal-to-noise ratio and a signal to contrast.

83. A system for analyzing an object, the system comprising:
a processing unit;
a first imaging device; and
a magnetic resonance probe, said magnetic resonance probe comprising a radiofrequency coil, designed and configured for generating a broad-band radiofrequency magnetic field, and a magnetic structure for generating a monotonic static magnetic field having a gradient, said magnetic structure comprising a plurality of domains, said plurality of domains being arranged within a volume having predetermined geometry, each of said plurality of domains being characterized by a predetermined and different magnetization vector, wherein said predetermined geometry and said plurality of domains are selected so as to generate said monotonic static magnetic field.

84. The system of claim 83, wherein the object is an internal object and the system is an invasive system.

85. The system of claim 83, wherein the object is an external object and the system a non-invasive system.

86. The system of claim, 83 wherein said first imaging device is an optical imaging device.

87. The system of claim 86, wherein said optical imaging device is a camera.

88. The system of claim 83, wherein said first imaging device is an ultra-sonic imaging device.

89. The system of claim 83, wherein said first imaging device is a nuclear medicine device, being sensitive to radioactive radiation.

90. The system of claim 83, further comprising at least one additional imaging device, said at least one additional imaging device is selected from the group consisting of an optical imaging device, a US imaging device and a nuclear medicine device.

91. The system of claim 83, wherein said first imaging device has a sufficiently wide field-of-view so as to surround at least a portion of said magnetic resonance probe.

92. The system of claim 83, further comprising a communication cable connected to said first imaging device.

93. The system of claim 83, further comprising at least one supporting device for supporting said magnetic resonance probe and said first imaging device.

94. The system of claim 83, further comprising a position tracking system for determining a position of said magnetic resonance probe.

95. The system of claim 94, wherein said position tracking system is selected from the group consisting of an articulated arm position tracking system, an accelerometers based position tracking system, a potentiometers based position tracking system, a sound wave based position tracking system, a radio frequency based position tracking system, an AC based position tracking system, a magnetic field based position tracking system and an optical based position tracking system.

96. The system of claim 83, wherein a size of said first imaging device and a size of said magnetic resonance probe are selected so as to allow said first imaging device and said magnetic resonance probe to be inserted into a body of a subject by endoscopy.

97. The system of claim 90, wherein a size of said first imaging device, a size of said magnetic resonance probe and sizes of said at least one imaging device are selected so as to allow said first imaging device, said magnetic resonance probe and said at least one imaging device to be inserted into a body of a subject by endoscopy.

98. The system of claim 83, further comprising a first gradient coil for generating a magnetic field having a gradient substantially in a first transverse direction.

99. The system of claim 98, further comprising a second gradient coil for generating a magnetic field having a gradient substantially in a second transverse direction.

100. The system of claim 98, wherein said first gradient coil is positioned on a surface of said magnetic structure.

101. The system of claim 99, wherein said first and said second gradient coils are positioned on a surface of said magnetic structure.

102. The system of claim 99, wherein said first and said second gradient coils are arranged in one layer.

103. The system of claim 99, wherein said first and said second gradient coils are arranged separate layers.

104. The system of claim 98, wherein said radiofrequency coil is positioned on a surface of said magnetic structure and further wherein said first gradient coil and said radiofrequency coil are arranged in one layer.

105. The system of claim 99 wherein said radiofrequency coil is positioned on a surface of said magnetic structure and further wherein said first gradient coil and said radiofrequency coil are arranged in one layer.

106. The system of claim 98, wherein said magnetic structure is detachable.

107. The system of claim 98, wherein said magnetic structure is replaceable.

108. The system of claim 98, wherein said magnetic structure comprises at least two parts, each independently operable to rotate about a longitudinal axis.

109. The system of claim 98, wherein said magnetic structure comprises at least two parts, each independently operable to move along a longitudinal axis.

110. The system of claim 83, wherein said plurality of domains comprises at least three domains.

111. The system of claim 83, wherein said plurality of domains comprises at least four domains.

112. The system of claim 83, wherein said predetermined geometry is selected from the group consisting of a cylinder, a disk, a prism, a sphere, a hemisphere, a portion of a sphere, an ellipsoid, a portion of ellipsoid, a hyperboloid, a portion of a hyperboloid, a paraboloid, a portion of a paraboloid, a cylindrical shell, a portion of a cylindrical shell, a polyhedron shell and a portion of a polyhedron shell.

113. The system of claim 83, wherein said predetermined geometry is elongated with respect to a longitudinal axis, and further wherein a size of said magnetic structure is selected so as to allow said magnetic structure to be inserted into a body of a subject by endoscopy.

114. The system of claim 113, wherein said plurality of domains are arranged along said longitudinal axis, and further wherein a magnetization vector of each domain has a component directed perpendicular to said longitudinal axis so that said monotonic static magnetic field is also in a direction perpendicular to said longitudinal axis.

115. The system of claim 113, wherein said plurality of domains are arranged along said longitudinal axis, and further wherein a magnetization vector of each domain has a component directed parallel to said longitudinal axis so that said monotonic static magnetic field is also in a direction parallel to said longitudinal axis.

116. The system of claim 113, further comprising at least one additional magnetic structure designed connectable to said magnetic structure, wherein said at least one additional magnetic structure capable of generating a monotonic static magnetic field.

117. The system of claim 116, wherein said at least one additional magnetic structure operable to rotate about a transverse axis by one of a plurality of predetermined angles.

118. The system of claim 113, wherein said monotonic static magnetic field varies along said longitudinal axis.

119. The system of claim 113, wherein said magnetization vectors vary along a radial direction, said radial direction being perpendicular to said longitudinal

120. The system of claim 113, wherein said magnetization vectors vary along an azimuthal direction, said azimuthal direction being perpendicular to said longitudinal axis.

121. The system of claim 83, said magnetic resonance probe further comprising at least one non-magnetic domain located so as to optimize a profile of said monotonic static magnetic field.

122. The system of claim 121, wherein said radiofrequency coil comprises a radiofrequency antenna and further wherein said at least one non-magnetic domain is constructed and designed for minimizing a load on said radiofrequency antenna and for minimizing magnetic acoustic ringing.

123. The system of claim 121, wherein a size of said at least one non-magnetic domain is selected so as to be surrounded by at least one radiofrequency coil.

124. The system of claim 121, wherein a size of said at least one non-magnetic domain is selected so as to minimize an amount of magnetic material present in said magnetic structure.

125. The system of claim 121, wherein said at least one radiofrequency coil comprises a soft magnetic material.

126. The system of claim 83, wherein said predetermined geometry is characterized by at least one substantially planar surface, said at least one substantially planar surface defined by an axis being perpendicularly to said, at least one substantially planar surface.

127. The system of claim 126, wherein said plurality of domains are concentrically arranged about a center of said magnetic structure, and further wherein a magnetization vector of each domain has a component directed along said axis, so that said monotonic static magnetic field is also directed along said axis.

128. The system of claim 83, wherein said predetermined, geometry is characterized by a planar surface and a non-planar surface, said non-planar surface having a first open end, adjacent to said planar surface, and a second open end, far from said planar surface, where an area of said first open end is smaller than an area of said second open end.

129. The system of claim 128, wherein said plurality of domains are arranged within said non-planar surface, and further wherein a magnetization vector of each domain has a component directed substantially parallel to said planar surface, so that said monotonic static magnetic field is also directed substantially parallel to said planar surface.

130. The system of claim 83, wherein said predetermined geometry is a shell having a cavity and a symmetry axis, said shell is selected from the group consisting of a hemisphere, a portion of a sphere, an ellipsoid, a portion of ellipsoid, a hyperboloid, a portion of a hyperboloid, a paraboloid, a portion of a paraboloid, a cylindrical shell, a portion of a cylindrical shell, a polyhedron shell and a portion of a polyhedron shell.

131. The system of claim 130, wherein a magnetization vector of each domain is directed along said symmetry axis so that said monotonic static magnetic field is also directed along said symmetry axis.

132. The system of claim 130, wherein a magnetization vector of each domain is directed perpendicularly to said symmetry axis so that said monotonic static magnetic field is also directed perpendicularly to said symmetry axis.

133. The system of claim 83, farther comprising at least one additional radiofrequency coil for generating a broad-band radiofrequency magnetic field and at least one additional magnetic structure for generating a monotonic static magnetic field having a gradient, wherein each of said at least one additional radiofrequency coil is in proximity to said at least one additional magnetic structure.

134. The system of claim 83, wherein the object is a mammal.

135. The system of claim 83, wherein the object is a organ of a mammal.

136. The system of claim 83, wherein the object is a tissue.

137. The system of claim 83, wherein the object is a swollen elastomer.

138. The system of claim 83, wherein the object is a food material.

139. The system of claim 83, wherein the object is liquid.

140. The system of claim 139, wherein said liquid is oil.

141. The system of claim 83, wherein the object is at least one type of molecules present in a solvent.

142. The system of claim 141, wherein said at least one type of molecules present in said solvent is selected from the group consisting of molecule dissolved in said solvent, a molecule dispersed in said solvent and a molecule emulsed in said solvent.

143. The system of claim 83, wherein said magnetic structure and said radiofrequency coil are designed and constructed so that said monotonic static magnetic field and said radiofrequency magnetic field are capable of generating predetermined and different magnetic resonance responses in predetermined and different types of cells.

144. The system of claim 143, wherein said predetermined types of cells are selected from the group consisting of a part of a tumor, a part of a malignant tumor, a part of a blood vessel tissue, a part of a pathological tissue and a part of a restenotic tissue.

145. The system of claim 83, wherein said magnetic structure and said radiofrequency coil are designed and constructed so that said monotonic static magnetic field and radiofrequency magnetic field are capable of generating a predetermined and different magnetic resonance responses in a first substance and in at least one additional substance present in or surrounded by said first substance, thereby distinguishing between said first substance and said at least one additional substance.

146. The system of claim 83, wherein said magnetic structure and said radiofrequency coil are designed and constructed so that said monotonic static magnetic field and said radiofrequency magnetic field are capable of generating a magnetic resonance response in at least one substance having dynamical resonance characteristics, said dynamical resonance characteristics being time-dependent.

147. The system of claim 146, design and constructed to monitor said dynamical resonance characteristics.

148. A system for analyzing an object, the system comprising:
a processing unit;
a magnetic resonance probe; and
a position tracking system for determining a position of said magnetic resonance probe;
wherein said magnetic resonance probe comprising a radiofrequency coil, designed and configured for generating a broad-band radiofrequency magnetic field, and a magnetic structure for generating a monotonic static magnetic field having a gradient, said magnetic structure comprising a plurality of domains, said plurality of domains being arranged within a volume having predetermined geometry, each of said plurality of domains being characterized by a predetermined and different magnetization vector, wherein said predetermined geometry and said plurality of domains are selected so as to generate said monotonic static magnetic field.

149. The system of claim 148, wherein said position tracking system is selected from the group consisting of an articulated arm position tracking system, an accelerometers based position tracking system, a potentiometers based position tracking system, a sound wave based position tracking system, a radio frequency based position tracking system, an AC based position tracking system, a magnetic field based position tracking system and an optical based position tracking system.

150. The system of claim 148, further comprising a first gradient coil for generating a magnetic field having a gradient substantially in a first transverse direction.

151. The system of claim 150, further comprising a second gradient coil for generating a magnetic field having a gradient substantially in a second transverse direction.

152. The system of claim 150, wherein said first gradient coil is positioned on a surface of said magnetic structure.

153. The system of claim 151, wherein said first and said second gradient coils are positioned on a surface of said magnetic structure.

154. The system of claim 151, wherein said first and said second gradient coils are arranged in one layer.

155. The system of claim 151, wherein said first and said second gradient coils are arranged separate layers.

156. The system of claim 150, wherein said radiofrequency coil is positioned on a surface of said magnetic structure and further wherein said first gradient coil and said radiofrequency coil are arranged in one layer.

157. The system of claim 151, wherein said radiofrequency coil is positioned on a surface of said magnetic structure and further wherein said first gradient coil and said radiofrequency coil are arranged in one layer.

158. The system of claim 150, wherein said magnetic structure is detachable.

159. The system of claim 150, wherein said magnetic structure is replaceable.

160. The system of claim 150, wherein said magnetic structure comprises at least two parts, each independently operable to rotate about a longitudinal axis.

161. The system of claim 150, wherein said magnetic structure comprises at least two parts, each independently operable to move along a longitudinal axis.

162. The system of claim 148, wherein said plurality of domains comprises at least three domains.

163. The system of claim 148, wherein said plurality of domains comprises at least four domains.

164. The system of claim 148, wherein said predetermined geometry is selected from the group consisting of a cylinder, a disk, a prism, a sphere, a hemisphere, a portion of a sphere, an ellipsoid, a portion of ellipsoid, a hyperboloid, a portion of a hyperboloid, a paraboloid, a portion of a paraboloid, a cylindrical shell, a portion of a cylindrical shell, a polyhedron shell and a portion of a polyhedron shell.

165. The system of claim 148, wherein said predetermined geometry is elongated with respect to a longitudinal axis.

166. The system of claim 165, wherein said plurality of domains are arranged along said longitudinal axis, and further wherein a magnetization vector of each domain has a component directed perpendicular to said longitudinal axis so that said monotonic static magnetic field is also in a direction perpendicular to said longitudinal axis.

167. The system of claim 165, wherein said plurality of domains are arranged along said longitudinal axis and further wherein a magnetization vector of each domain has a component directed parallel to said longitudinal axis so that said monotonic static magnetic field is also in a direction on parallel to said longitudinal axis.

168. The system of claim 165, further comprising at least one additional magnetic structure designed connectable to said magnetic structure, wherein said at least one additional magnetic structure capable of generating a monotonic static magnetic field.

169. The system of claim 168, wherein said at least one additional magnetic structure operable to rotate about a transverse axis by one of a plurality of predetermined angles.

170. The system of claim 165, wherein said monotonic static magnetic field varies along said longitudinal axis.

171. The system of claim 165, wherein said magnetization vectors vary along a radial direction, said radial direction being perpendicular to said longitudinal axis.

172. The system of claim 165, wherein said magnetization vectors vary along an azimuthal direction, said azimuthal direction being perpendicular to said longitudinal axis.

173. The system of claim 148, further comprising at least one non-magnetic domain located so as to optimize a profile of said monotonic static magnetic field.

174. The system of claim 173, wherein said radiofrequency coil comprises a radiofrequency antenna and further wherein said at least one non-magnetic domain is constructed and designed for minimizing a load on said radiofrequency antenna and for minimizing magnetic acoustic ringing.

175. The system of claim 173, wherein a size of said at least one non-magnetic domain is selected so as to be surrounded by at least one radiofrequency coil.

176. The system of claim 173, wherein a size of said at least one non-magnetic domain is selected so as to minimize an amount of magnetic material present in said magnetic structure.

177. The system of claim 173, wherein said at least one radiofrequency coil comprises a soft magnetic material.

178. The system of claim 148, wherein said predetermined geometry is characterized by at least one substantially planar surface, said at least one substantially planar surface defined by an axis being perpendicularly to said at least one substantially planar surface.

179. The system of claim 178, wherein said plurality of domains are concentrically arranged about a center of said magnetic structure, and further wherein a magnetization vector of each domain has a component directed along said axis, so that said monotonic static magnetic field is also directed along said axis.

180. The system of claim 148, wherein said predetermined geometry is characterized by a planar surface and a non-planar surface, said non-planar surface having a first open end, adjacent to said planar surface, and a second open end, far from said planar surface, where an area of said first open end is smaller than an area of said second open end.

181. The system of claim 180, wherein said plurality of domains are arranged within said non-planar surface, and further wherein a magnetization vector of each domain has a component directed substantially parallel to said planar surface, so that said monotonic static magnetic field is also directed substantially parallel to said planar surface.

182. The system of claim 148, wherein said predetermined geometry is a shell having a cavity and asymmetry axis, said shell is selected from the group consisting of a hemisphere, a portion of a sphere, an ellipsoids, a portion of ellipsoid, a hyperboloid, a portion of a hyperboloid, a paraboloid, a portion of a paraboloid, a cylindrical shell, a portion of a, cylindrical shell, a polyhedron shell and a portion of a polyhedron shell.

183. The system of claim 182, wherein a magnetization vector of each domain is directed along said symmetry axis so that said monotonic static magnetic field is also directed along said symmetry axis.

184. The system of claim 182, wherein a magnetization vector of each domain is directed perpendicularly to said symmetry axis so that said monotonic static magnetic field is also directed perpendicularly to said symmetry axis.

185. The system of claim 148, further comprising at least one additional radiofrequency coil for generating a broad-band radiofrequency magnetic field and at least one additional magnetic structure for generating a monotonic static magnetic field having a gradient, wherein each of said at least one additional radiofrequency coil is in proximity to said at least one additional magnetic structure.

186. The system of claim 148, wherein the object is a mammal.

187. The system of claim 148, wherein the object is an organ of a mammal.

188. The system of claim 148, wherein the object is a tissue.

189. The system of claim 148, wherein the object is a swollen elastomer.

190. The system of claim 148, wherein the object is a food material.

191. The system of claim 148, wherein the object is liquid.

192. The system of claim 191, wherein said liquid is oil.

193. The system of claim 148, wherein the object is at least one type of molecules present in a solvent.

194. The system of claim 193, wherein said a least one type of molecules present in said solvent is selected from the group consisting of molecule dissolved in said solvent, a molecule dispersed in said solvent and a molecule emulsed in said solvent.

195. The system of claim 148, wherein said magnetic structure and said radiofrequency coil are designed and constructed so that said monotonic static magnetic field and said radiofrequency magnetic field are capable of generating predetermined and different magnetic resonance responses in predetermined and different types of cells.

196. The system of claim 195, wherein said predetermined types of cells are selected from the group consisting of a part of a tumor, a part of a malignant tumor, a part of a blood vessel tissue, a part of a pathological tissue and a part of a restenotic tissue.

197. The system of claim 148, wherein said magnetic structure and said radiofrequency coil are designed and constructed so that said monotonic static magnetic field and said radiofrequency magnetic field are capable of generating a predetermined and different magnetic resonance responses in a first substance and in at least one additional substance present in or surrounded by said first substance, thereby distinguishing between said first substance and said at least one additional substance.

198. The system of claim 148, wherein said magnetic structure and said radiofrequency coil are designed and constructed so that said monotonic static magnetic field and said radiofrequency magnetic field are capable of generating a magnetic resonance response in at least one substance having dynamical resonance characteristics, said dynamical resonance characteristics being time-dependent.

199. The system of claim 198, design and constructed to monitor said dynamical resonance characteristics.
